(19) United States
(12) Patent Application Publication
(10) Pub. No.: US 2013/0014288 A1
(43) Pub. Date: Jan. 10, 2013

(54) NOVEL REPORTER-TAGGED RECOMBINANT MEMBRANE PROTEINS WITH TRANSMEMBRANE LINKERS

(75) Inventors: Jonathan W. Jarvik, Pittsburgh, PA (US); John P. Holleran, Allison Park, PA (US)

(73) Assignee: Carnegie Mellon University, Pittsburgh, PA (US)

(21) Appl. No.: 13/490,353

(22) Filed: Jun. 6, 2012

Related U.S. Application Data
(60) Provisional application No. 61/520,181, filed on Jun. 6, 2011.

(51) Int. Cl. C07K 19/00; A01K 67/00; C12N 5/10; C12N 9/96; C12N 15/62

(52) U.S. Cl. 800/13; 435/188; 536/23.4; 536/23.2; 435/357; 435/369; 530/350

(57) ABSTRACT

Recombinant protein constructs are described that comprises a membrane protein whose N- or C-terminus in the native state is recombinantly linked through a membrane-spanning linker polypeptide to a reporter polypeptide. The reporter polypeptide may be a fluorogen activating protein capable of binding a fluorogen to detect the location and relative abundance of the membrane protein, and more specifically to detect protein trafficking to the cell surface using a cell impermeant fluorogen probe.

### CROSS-REFERENCE TO RELATED APPLICATIONS

This application claims the benefit of U.S. Provisional Application Ser. No. 61/520,181 filed Jun. 6, 2011, the contents of which are hereby incorporated herein by reference.

### GOVERNMENT RIGHTS

This invention was made with government support under National Center for Research Resources, National Institutes of Health; grant U54 RR022241, NIH grant P30 DK072506, DK68196. The government has certain rights in this invention.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### REFERENCE TO SEQUENCE LISTING

This Application contains a Sequence Listing in accordance with 37 C.F.R. §§1.821-1.825. The material in the Sequence Listing text file is herein incorporated by reference in its entirety in accordance with 37 C.F.R. §1.52(e) (5). The Sequence Listing, entitled “110455 Sequence Listing_ST25.txt”, contains one 56 Kb text file and was created on Jun. 6, 2012 using an IBM-PC machine format.

### BACKGROUND

### Field of the Invention

The invention relates to reporter-tagged recombinant membrane proteins and methods for making and using such proteins.

Membrane proteins play roles in thousands of cellular processes and are the targets of more than half of all therapeutic drugs. Numerous membrane proteins exhibit regulated translocation between the cell surface and the cell interior. For example, most G-protein coupled receptors (GPCRs) and receptor tyrosine kinases (RTKs) are internalized by endocytosis after exposure to agonists (see, Kohout and Lefkowitz, Regulation of G protein-coupled receptor kinases and arrestins during receptor desensitization, Molecular pharmacology, 2003; Bache et al., Defective downregulation of receptor tyrosine kinases in cancer, The EMBO journal, 2004). Translocation between the cell interior and the membrane is also important for numerous ion and metabolite transporters that traffic to the membrane in response to particular physiological stimuli. (Ma and Jan, ER transport signals and trafficking of potassium channels and receptors, Current opinion in neurobiology, 2002).

The topology of almost all membrane proteins is such that one or both termini are located in the cytosol. Thus the GPCRs and RTKs have cytosolic C-termini, and the ABC transporters, and solute carrier proteins (SLCs) have cytosolic N- and C-termini. (Almen et al., Mapping the human membrane proteome: a majority of the human membrane proteins can be classified according to function and evolutionary origin, BMC biology, 2009).

To monitor and assay a particular membrane protein in cells and tissues, it is often advantageous to express in the cells or tissues a recombinant fusion protein comprised of the protein of interest fused at its N- or C-terminus to a reporter polypeptide. Reporter polypeptides take many forms, including epitope tags such as cMyc or FLAG, fluorescent or luminescent proteins such as GFP or luciferase, enzymes such as beta-galactosidase or alkaline phosphatase, and fluorogen-activating proteins (FAPs) such as MG-13 or AM2-2. (Szent-Gyorgyi et al., Fluorogen-activating single-chain antibodies for imaging cell surface proteins, Nature biotechnology, 2008).

A deficiency with the use of reporter-tagged membrane proteins with the structures described above is that the reporter may be on the wrong side of the membrane for it to function as needed. Thus, for example, the tetracysteine-dependent FIAsH and ReAsH reagents do not work in the extracellular oxidizing environment and so for proteins with extracellular N-termini they cannot be used to detect recombinant proteins with N-terminal tetracysteine tags. Likewise, certain FAP reporters do not work in the reducing environment of the cytosol and thus cannot be used to detect many membrane proteins that are FAP-tagged at their N- or C-termini. Further, and as discussed in greater detail below, certain FAP-based assays for membrane protein translocation depend on having the FAP on the extracellular side of the plasma membrane. If the membrane protein of interest has cytosolic N- and C-termini—and this is the case for many medically important membrane proteins—these assays cannot be used when the proteins have been FAP-tagged at their N- or C-termini.

### SUMMARY OF THE INVENTION

The various embodiments of the invention overcome the deficiencies described above by providing a membrane-spanning linker domain between the reporter and the terminus of the protein of interest, thereby placing the reporter on the opposite side of the membrane from the native terminus, where the reporter can function as needed.

Embodiments of the invention described herein include recombinant protein constructs each comprising a membrane-protein whose terminus in its native state is normally cytosolic, a fusion partner comprising a reporter polypeptide, and a membrane-spanning linker bound at a first end to the cytosolic terminus of the membrane protein and at a second end to the reporter polypeptide.

The membrane-protein may be any naturally occurring membrane-protein or any modified version of a natural membrane protein. In various embodiments, the membrane protein may be associated with a target biological function or malfunction, such as, for example, the membrane proteins B₂AR, GLUT4 and CFTR. Embodiments of the membrane protein may be those having in the native state, a cytosolic N-terminus and the membrane-spanning linker may be recombinantly linked to the N-terminus. Embodiments of the membrane protein may be those having in the native state, a cytosolic C-terminus and the membrane-spanning linker may be recombinantly linked to the C-terminus.

In various embodiments, the reporter polypeptide may be a fluorescent or luminescent protein, a fluorogen activating protein (FAP), or another reporter polypeptide. Exemplary fluorescent and luminescent proteins include GFP or luciferase, and enzymes such as beta-galactosidase or alkaline phosphatase. Exemplary FAPs include those selected from the group consisting of HL1-TO1 (SEQ. ID. NO.: 33), HL1.1-TO1 (SEQ. ID. NO.: 34), HL1.0.1-TO1 (SEQ. ID. NO.: 26), HL4-MG (SEQ. ID. NO.: 35), L5-MG (SEQ. ID. NO.: 36), H6-MG (SEQ. ID. NO.: 37), K7 (SEQ. ID. NO.: 38), M8 (SEQ. ID. NO.: 39) and dNP138 (SEQ. ID. NO.: 40). Other reporter polypeptides include epitope tags such as cMyc or FLAG.

The membrane-spanning linker may be a transmembrane spanning alpha helical amino acid sequence derived from a known membrane protein such as human platelet derived growth factor receptor (PDGFR) or may be a synthetic sequence known to form a membrane-spanning domain. In various embodiments, the membrane-spanning linker may have at least 85%, at least about 90%, at least about 95%, about 96%, about 97%, about 98%, about 99% or about 100% sequence identity to a transmembrane domain of human platelet derived growth factor receptor beta (PDGFRB) protein. In various embodiments, the membrane-spanning linker may have at least 85%, at least about 90%, at least about 95%, about 96%, about 97%, about 98%, about 99% or about 100% sequence identity to a transmembrane domain selected from the group consisting of CD4TL, EGFR, VEGFR1, VGFR2, VGFR3, FGFR1-4, FGFR6 and TGFBR1-3. In various embodiments, the membrane-spanning linker may be appended to the N- or the C-terminus of the membrane protein.

In other embodiments, the membrane spanning linker may have at least 85%, at least about 90%, at least about 95%, about 96%, about 97%, about 98%, about 99% or about 100% sequence identity to any desired domain of a transmembrane protein. It will be apparent from the examples provided below that one may select a desired transmembrane protein or a fragment thereof, and use the nucleotides that encode for the desired protein or fragment in the methods described herein to form recombinant protein constructs according to the invention.

In certain aspects of the invention, the recombinant protein construct may include as the membrane protein, one of human GLUT4 or human CFTR, and as the reporter polypeptide, a fluorogen activating protein selected from one of HL1.1-TO1 or HL4-MG.

In certain aspects of the invention, the recombinant protein construct may include in addition to one of these membrane proteins and reporter polypeptides, a membrane spanning linker selected from PDGFR, CD4TL, EGFR, VEGFR1, VGFR2, VGFR3, FGFR1-4, and TGFBR1-3.

In various embodiments, a recombinant polypeptide is described herein that allows a cytosolic terminus of a membrane protein to be tagged with a reporter whose utility requires that it be located on the opposite side of the membrane. When, for example, a FAP-tagged protein is used in a surface fluorescence depletion assay or an internal fluorescence accumulation assay (Fisher et al., Detection and quantification of beta2AR internalization in living cells using FAP-based biosensor technology, Journal of biomolecular screening, 2010), the tag must be non-cytosolic.

In various embodiments that employ FAPs as reporter polypeptides, the approach described herein enables specific detection of the fusion protein by the addition of a single soluble reagent, with no subsequent wash or separation steps. For example, direct detection of F508de1-CFTR, described more fully below, at the cell surface with a single labeling step is shown herein to improve throughput and increase the dynamic range of corrector screening assays.

In certain embodiments, membrane proteins are provided with reporter protein-tagged non-cytosolic termini in place of native cytosolic termini.

Various embodiments described herein make use of the genetically encoded biosensor system recently described in Szent-Gyorgyi C, et al., Fluorogen-activating single-chain antibodies for imaging cell surface proteins, Nature biotechnology, 2008. and U.S. Patent Application Publication US-2011-0159519-A1, both of which are hereby incorporated herein by reference. In this genetically encoded biosensor system, fluorescent signal depends on the interaction between a reporter polypeptide and a small molecule (the fluorogen) that is only fluorescent when bound to the reporter polypeptide. These polypeptides, which were selected from a library of single chain antibodies (scFv's) and improved by directed evolution, were given the name FAPs, for fluorogen activating proteins. The FAP system consists of a genetically encoded FAP and chemical label, the fluorogen. Each FAP has specificity to a particular fluorogen. The HL4-MG FAP, for example, is capable of activating malachite green fluorogens, cell impermeable MG-11P, and cell permeable MG-ester, and the HL1.1-TO1 FAP activates the cell impermeable thiazole orange fluorogen, TO1-2P. When incorporated into proteins, FAP domains provide a binding site specific to cognate fluorogens that, when bound, report protein location and abundance in time and space. Fluorescence signal is generated only upon addition of a second component (e.g., the fluorogen). FAPs can be visualized directly after fluorogen addition on a time scale of seconds to minutes. Fluorescence visualization can also be spatially controlled by the appropriate choice of fluorogen, enabling one to selectively observe proteins at particular cellular locations.

In certain aspects, the invention may comprise a nucleic acid molecule encoding any of the recombinant protein constructs encompassed by the disclosure herein. In certain aspects, the invention may comprise a cell comprising any of the recombinant protein constructs encompassed by the disclosure herein. In certain aspects, the invention may comprise a cell comprising any of the nucleic acid molecules encoding any of the recombinant protein constructs encompassed by the disclosure herein. In certain embodiments, the invention may comprise a transgenic nonhuman animal having a plurality of cells comprising any of the recombinant protein constructs encompassed by the disclosure herein.

When fused to an extracellular terminus and visualized using membrane-impermeant fluorogen, FAP reporter polypeptides provide an opportunity to selectively observe only those protein molecules that are present in the plasma membrane, with molecules elsewhere in the cell remaining dark because they cannot contact the fluorogen. This has permitted, for example, direct detection of F508de1-CFTR at the cell surface with a single labeling step, described more fully below, that is shown herein to improve throughput and increase the dynamic range of corrector screening assays. Thus, in certain aspects, the invention may include an assay for detecting the presence of a membrane protein of interest. Such assays take advantage of membrane-impermeant fluorogens that are added to the extracellular medium where they contact and label individual FAP polypeptides that are also extracellular, but do not contact and label FAP polypeptides units that are intracellular or cytosolic.

### BRIEF DESCRIPTION OF FIGURES

Various features and characteristics of the non-limiting and non-exhaustive embodiments disclosed and described in this specification may be better understood by reference to the accompanying figures, in which:

FIGS. 1A-C are schematic illustrations of a native membrane protein having cytosolic N- and C-termini. FIGS. 1B and C illustrate embodiments of the recombinant protein construct described herein showing a membrane protein (A) having in its native state a cytosolic N-terminus (“inside”) and a cytosolic C-terminus (“inside”), an extracellular reporter and a membrane spanning linker between the membrane protein native N- or C-terminus, respectively, and the extracellular reporter. FIG. 1C further illustrates schematically the embodiments wherein the reporter is positioned either within the sequence of the membrane spanning linker or between first and second membrane spanning linkers, where the second linker extends from the extracellular reporter to the cytosol to reposition a C-terminus in the cytosol.

FIG. 2 is a schematic representation of the expression unit in the pDisplaySacLac2 and pBabeSacLac2 vectors. From 5′ to 3′, the unit begins at ATG start codon followed by the murine lgκ leader sequence and an HA epitope tag. Next, is the bacterial selection marker SacB flanked by Sfil restriction sites, followed by a c-Myc epitope. This is followed by a membrane spanning segment derived from the human platelet derived growth factor receptor (PDGFR) gene, and then a LacZA gene flanked by Pflml restriction sites.

FIGS. 3A-C show topological schematic images with respect to the plasma membrane, where the top is extracellular and the bottom is intracellular, and the corresponding fluorescence images of recombinant protein constructs in NIH 3T3 cells. FIG. 3A represents the topology and fluorescence channels A, B, and C in different colors (e.g., green (A), red (B), and a merger of A and B (C)) of an embodiment of a fusion protein, HL1.1(TO1 2P) with the mRFP membrane protein. FIG. 3B represents the topology and fluorescence channels E, F, and G in different colors (e.g., green (E), red (F), and a merger of E and F (G)) of another embodiment of a fusion protein, HL4(MG 11P) with the EGFP membrane protein. FIG. 3C represents the topology and fluorescence channels I, J, and K in different colors (e.g., green (I), red (J), and a merger of I and J (K)) of another embodiment of a fusion protein, HL4(MG ester) with the EGFP membrane protein. FIGS. 3A-3B are FAP-FP expressing cells in the presence of impermeable fluorogen. FIG. 3C represent cell permeable fluorogen labeling of FAP-FP cells. Scale bars represent 10 μm.

FIGS. 4A-C are histograms of the fluorescent images of FIGS. 3A-C, respectively, wherein FIG. 4A shows a histogram generated from the fluorescence images of FIG. 3A (A-C) by plotting the pixel intensity across a specified region (indicated by the white line in the fluorescence images); FIG. 4B shows a histogram generated from the fluorescence images FIG. 3B (E-G) by plotting the pixel intensity across a specified region (indicated by the white line in the fluorescence images); and FIG. 4C shows a histogram generated from the fluorescence images 3C (I-K) by plotting the pixel intensity across a specified region (indicated by the white line in the fluorescence images).

FIG. 5 shows fluorescence images of HL4-MG and EGFP fusions to transmembrane proteins. The left column shows representations of fusion protein topology in the plasma membrane, where the top is extracellular and the bottom is intracellular. Fluorescent images (A-C) show NIH 3T3 cells expressing human B₂AR with FAP (HL4 MG-11P and HL4 MG-ester), or EGFP fused to the extracellular N-terminus. Fluorescent images (D-F) show C2C12 cells expressing human GLUT4 with one of the extracellular FAPs or EGFP, plus new transmembrane domain, fused to the intracellular N-terminus of the membrane protein. Fluorescent images (G-I) show HEK293 cells expressing CFTR with one of the extracellular FAPs or EGFP, plus new transmembrane domain, fused to the intracellular N-terminus of the membrane protein. Fluorescent images (A, D, G) show cells that were imaged in the presence of impermeable fluorogen, MG-11P. Fluorescent images (B, E, H) show that the membrane permeant fluorogen, MG-ester, labels intracellular fusion proteins. Scale bars represent 10 μm. Fluorescent images (C, F, I) show intracellular and surface labeling with the membrane permeant protein EGFP.

FIGS. 6A and B are fluorescent images by confocal microscopy showing the internalization of FAP-tagged B₂AR in response to agonist treatment. FIG. 6A represents cells expressing HL1.1-B₂AR that were incubated in membrane impermeant fluorogen TO1-2P. FIG. 6B represents cells expressing HL1.1-B₂AR that were incubated in membrane impermeant fluorogen TO1-2P plus isoproterenol. Scale represents 10 μm.

FIG. 7 represents the topology of cystic fibrosis transmembrane conditioning regulator (CFTR) with FAP reporter polypeptides, showing a schematic diagram of CFTR tagged with a FAP component at the N-terminus via an extra transmembrane spanning fragment (FAP-CFTR).

FIG. 8 represents the FAP detection of CFTR. HEK293 cells were imaged by confocal fluorescence microscopy. FAP-CFTR wild type (WT) was detected exclusively at the cell surface using the cell impermeant fluorogen, MG-11p (A) or at the cell surface and in intracellular compartments throughout the cell with cell permeant fluorogen, MG-ester (B). Scale bars are 10 μm.

FIG. 9 represents the correctors rescue of the trafficking defect of FAP CFTR F508de1. HEK293 cells stably expressing FAP-F508de1-CFTR (A-C) were imaged using confocal fluorescence microscopy. After treatment with the vehicle (DMSO) control for 24 hours, there was no staining observed in the presence of cell impermeant fluorogen, 50 nM MG-11p (A). A strong intracellular signal which was not localized to the plasma membrane was revealed upon addition of the cell permeant fluorogen, MG-ester (B). After treatment with a combination of correctors, C4 and C18 for 24 hours, F508de1-CFTR was detected at the cell surface using cell impermeant fluorogen, 50 nM MG-11p (C). Scale bars indicate 10 μm.

FIG. 10 represents the temperature rescue of FAP CFTR F508de1. Rescue of FAP F508de1-CFTR by low temperature was visualized by confocal fluorescence microscopy. HEK293 cells expressing FAP F508de1-CFTR constructs were incubated at the permissive temperature, 30° C., for 24 hours. Surface expression was confirmed by activation of the impermeant fluorogen, 50 nM MG-11p, for FAP-F508de1-CFTR at both the cell surface and in intracellular pools (B). Controls without low temperature were shown in FIG. 9A. Scale bars indicate 10 μm.

FIGS. 11A-B represent the correctors rescue F508de1-CFTR FAP Function using SPQ (6-methoxy-N-(3-sulfopropyl)quinolinium) fluorescence traces showing iodide efflux in response to 10 μM Fsk stimulation. FIG. 11B shows graphs of the mean fluorescence over time for stably expressing FAP-F508de1-CFTR cells that were treated with either the control vehicle (DMSO) or a combination of correctors, C4+C18 for 24 hours. Iodide efflux was stimulated with Fsk and a potentiator, 300 nM P2.

FIG. 12 represents the biochemical properties of FAP-CFTRs and evidence of corrector rescue. Immunoblotting was performed on whole cell lysates from cells expressing CFTR. FIG. 12A represents HEK293 cells expressing either untagged or FAP CFTR constructs. F508de1 constructs that received treatments with correctors CFFT-002+C18 or C4+C18 for 24 hours showed an accumulation of higher molecular weight bands that were not present in the vehicle treated control. FAP-F508de1-CFTR corrected lanes have acquired a fully glycosylated C band. FIG. 12B shows the densitometry analysis of these immunoblots showing decreasing molecular weight from left to right. Clear differences are observed between the combination of corrector treatment and the DMSO control vehicle.

FIGS. 13A and B represent the quantification of corrector efficacies of single corrector compounds or combinations by flow cytometry. FIG. 13A shows representative flow cytometry histograms for FAP-F508de1 and HEK293 cell lines showing the distribution of MG-11p fluorescence signal and the vehicle control (dashed line) corrector treatment. Fluorescence activity is plotted along the horizontal axis using a logarithmic scale. FIG. 13B is a bar graph showing the mean fluorescence intensity of HEK293 cells expressing FAP-F508de1-CFTR for each condition, normalized to the effect of C18.

FIG. 14 is a chart showing the structures for correctors C4, C18 and CFFT-002.

FIG. 15 is a chart showing the properties of three representative Florescent Activating Proteins (FAPs) and fluorogens and their structures. The fluorogens are derivatives of malachite green (MG) or thiazole orange (TO). The FAPs are comprised of hypervariable heavy (H) and light (L) chains joined by a flexible linker

FIG. 16 illustrates the structures for additional useful fluorogens.

FIG. 17 illustrates the amino acid sequence of an exemplary recombinant protein construct (SEQ. ID. NO.: 22) showing the sequences for the several sections that may be included therein.

### DETAILED DESCRIPTION OF THE INVENTION

Introduction. Various embodiments are described and illustrated in this specification to provide an overall understanding of the structure, function, operation, manufacture, and use of the disclosed compositions, systems, and methods. It is understood that the various embodiments described and illustrated in this specification are non-limiting and non-exhaustive. Thus, the invention is not limited by the description of the various non-limiting and non-exhaustive embodiments disclosed in this specification. The features and characteristics illustrated and/or described in connection with various embodiments may be combined with the features and characteristics of other embodiments. Such modifications and variations are intended to be included within the scope of this specification. As such, the claims may be amended to recite any features or characteristics expressly or inherently described in, or otherwise expressly or inherently supported by, this specification. The various embodiments disclosed and described in this specification can comprise, consist of, or consist essentially of, or be characterized by the features and characteristics as variously described herein.

Any patent, publication, or other disclosure material identified herein is incorporated by reference into this specification in its entirety unless otherwise indicated, but only to the extent that the incorporated material does not conflict with existing definitions, statements, or other disclosure material expressly set forth in this specification. As such, and to the extent necessary, the express disclosure as set forth in this specification supersedes any conflicting material incorporated by reference herein. Any material, or portion thereof, that is said to be incorporated by reference into this specification, but which conflicts with existing definitions, statements, or other disclosure material set forth herein, is only incorporated to the extent that no conflict arises between that incorporated material and the existing disclosure material. Applicant reserves the right to amend this specification to expressly recite any subject matter, or portion thereof, incorporated by reference herein.

Reference throughout this specification to “various non-limiting embodiments,” or the like, means that a particular feature or characteristic may be included in an embodiment. Thus, use of the phrase “in various non-limiting embodiments,” or the like, in this specification does not necessarily refer to a common embodiment, and may refer to different embodiments. Further, the particular features or characteristics may be combined in any suitable manner in one or more embodiments. Thus, the particular features or characteristics illustrated or described in connection with various embodiments may be combined, in whole or in part, with the features or characteristics of one or more other embodiments without limitation. Such modifications and variations are intended to be included within the scope of the present specification.

In this specification, other than where otherwise indicated, all numerical parameters are to be understood as being prefaced and modified in all instances by the term “about”, in which the numerical parameters possess the inherent variability characteristic of the underlying measurement techniques used to determine the numerical value of the parameter. At the very least, and not as an attempt to limit the application of the doctrine of equivalents to the scope of the claims, each numerical parameter described in the present description should at least be construed in light of the number of reported significant digits and by applying ordinary rounding techniques.

Also, any numerical range recited in this specification is intended to include all sub-ranges of the same numerical precision subsumed within the recited range. For example, a range of “1.0 to 10.0” is intended to include all sub-ranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited in this specification is intended to include all lower numerical limitations subsumed therein and any minimum numerical limitation recited in this specification is intended to include all higher numerical limitations subsumed therein. All such ranges are intended to be inherently described in this specification such that amending to expressly recite any such sub-ranges would comply with the applicable disclosure requirements.

The grammatical articles “one”, “a”, “an”, and “the”, as used in this specification, are intended to include “at least one” or “one or more”, unless otherwise indicated. Thus, the articles are used in this specification to refer to one or more than one (i.e., to “at least one”) of the grammatical objects of the article. By way of example, “a component” means one or more components, and thus, possibly, more than one component is contemplated and may be employed or used in an implementation of the described embodiments. Further, the use of a singular noun includes the plural, and the use of a plural noun includes the singular, unless the context of the usage requires otherwise.

“Bound,” “bind”, “binding”, “associated with”, or “attachment”, “attached to” and the like as used herein means covalent or non-covalent binding, including without limitation, the attractive intermolecular forces between two or more compounds, substituents, molecules, ions or atoms that may or may not involve sharing or donating electrons. Non-covalent interactions may include ionic bonds, hydrophobic interactions, hydrogen bonds, van der Waals forces (dispersion attractions, dipole-dipole and dipole-induced dipole interactions), intercalation, entropic forces, and chemical polarity.

As used herein, the term “non-cytosolic” refers to the extracellular side of the membrane for proteins located in the plasma membrane. For proteins located in the membranes of internal cellular compartments such as nuclei, endoplasmic reticulum, Golgi apparatus, lysosomes, endosomes, mitochondria, chloroplasts and peroxisomes, the term “non-cytosolic” refers to the side of the membrane facing the interior of the compartment.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing.

The term “ligand” refers to a binding moiety for a specific target molecule. The molecule can be a cognate receptor, a protein, a small molecule, a hapten, or any other relevant molecule.

The term “antibody” refers to an immunoglobulin, derivatives thereof which maintain specific binding ability, and proteins having a binding domain which is homologous or largely homologous to an immunoglobulin binding domain. As such, the antibody operates as a ligand for its cognate antigen. Ehrlich et al., U.S. Pat. No. 4,355,023.

The term “recombinantly linked” as used herein means that the components are part of the same polypeptide, said polypeptide being the translation product of a recombinant gene.

As used herein, the term “epitope” refers to a physical structure on a molecule that interacts with a selectivity component, such as an antibody. In exemplary embodiments, epitope refers to a desired region on a target molecule that specifically interacts with a selectivity component.

“Interact” is meant to include all detectable interactions between molecules, such as may be detected using, for example, a hybridization assay. Interact also includes “binding” interactions between molecules. Interactions may be, for example, protein-protein, protein-nucleic acid, protein-small molecule or small molecule-nucleic acid, and includes for example, antibody-antigen binding, receptor-ligand binding, hybridization, and other forms of binding. In certain embodiments, an interaction between a ligand and a specific target will lead to the formation of a complex, wherein the ligand and the target are unlikely to dissociate. Such affinity for a ligand and its target can be defined by the dissociation constant (K_{d}) as known in the art. A complex may include a ligand for a specific dye and is referred to herein as a “ligand-dye” complex.

As used herein, the terms “reporter,” “reporter protein,” and “reporter polypeptide” refer to a polypeptide with properties that allow one to detect its presence in whole organisms, tissues, cells, or mixtures of molecules prepared from tissues or cells. Examples of reporters include, but are not limited to, the following: enzymes such as beta-galactosidase or alkaline phosphatase that can be detected using chromogenic or fluorogenic substrates, fluorescent proteins such as GFP or RFP, luminescent proteins such as firefly or Renilla luciferase, epitope tags such as cMyc or FLAG or 6× His, biotinylation acceptor sequences such as AviTag, proteins that bind small molecules covalently such as SNAP-tag and HALO-tag or non-covalently such as glutathione S-transferase, and fluorogen-activating proteins (FAPs) such as MG13 (sometimes referred to as HL4), dNP-138 (sometimes referred to as L5-MG-dimer) or AM2.2 (sometimes referred to as HL1.0.1). Some FAPs are single chain scFVs. Other FAPs are dimers. The nomenclatue used to designate the various FAPs include the letters H, L or HL, wherein HL signifies heavy and light chains; L signifies light chain only, and H signifies heavy chain only.

The term “membrane-spanning linker” refers to a transmembrane spanning alpha helical amino acid sequence derived from a known membrane protein, for example the membrane-spanning domain from the human platelet derived growth factor receptor (PDGFR). A membrane spanning linker may also be a synthetic amino acid sequence that is known to form a membrane-spanning domain when present in a protein.

A “fusion protein” or “fusion polypeptide” refers to a recombinant chimeric protein as that term is known in the art and may be constructed using methods known in the art.

The term “fusion partner” refers to an individual protein unit in a fusion protein. In many examples of fusion proteins, one of the fusion partners is a reporter protein

“Isolated”, with respect to nucleic acids, such as DNA or RNA, refers to molecules separated from other DNAs, or RNAs, respectively that are present in the natural source of the macromolecule. Isolated also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. “Isolated” also refers to polypeptides which are isolated from other cellular proteins and is meant to encompass both purified and recombinant polypeptides.

The term “nucleic acid” refers to a polymeric form of nucleotides, either ribonucleotides or deoxynucleotides or a modified form of either type of nucleotide. The terms should also be understood to include, as equivalents, analogs of either RNA or DNA made from nucleotide analogs, and, as applicable to the embodiment being described, single-stranded (such as sense or antisense) and double-stranded polynucleotides.

The term “polypeptide”, and the terms “protein” and “peptide” which are used interchangeably herein, refers to a polymer of amino acids.

The terms “polypeptide fragment” or “fragment”, when used in regards to a reference polypeptide, refers to a polypeptide in which amino acid residues are deleted as compared to the reference polypeptide itself, but where the remaining amino acid sequence is usually identical to the corresponding positions in the reference polypeptide. Such deletions may occur at the amino-terminus or carboxy-terminus of the reference polypeptide, or alternatively both. Fragments typically are at least 5, 10, 20, 50, 100, 500 or more amino acids long. A fragment can retain one or more of the biological activities of the reference polypeptide.

The term “sequence homology” refers to the proportion of base matches between two nucleic acid sequences or the proportion of amino acid matches between two amino acid sequences. When sequence homology is expressed as a percentage, e.g., 50%, the percentage denotes the proportion of matches over the length of sequence from a desired sequence (e.g., SEQ. ID NO: 22) that is compared to some other sequence. Gaps (in either of the two sequences) are permitted to maximize matching; gap lengths of 15 bases or less are usually used, 6 bases or less are used more frequently, with 2 bases or less used even more frequently. The term “sequence identity” means that sequences are identical (i.e., on a nucleotide-by-nucleotide basis for nucleic acids or amino acid-by-amino acid basis for polypeptides) over a window of comparison. The term “percentage of sequence identity” is calculated by comparing two optimally aligned sequences over the comparison window, determining the number of positions at which the identical amino acids occurs in both sequences to yield the number of matched positions, dividing the number of matched positions by the total number of positions in the comparison window, and multiplying the result by 100 to yield the percentage of sequence identity. Methods to calculate sequence identity are known to those of skill in the art.

Recombinant Constructs. The recombinant protein constructs described herein comprise a fusion partner in the form of a reporter polypeptide, a membrane protein, and a membrane spanning linker positioned between the reporter polypeptide and the membrane protein. Such recombinant proteins provide, among other things, means for selectively visualizing membrane proteins and for monitoring the trafficking of these proteins to and from the cell or organelle surface.

The recombinant fluorogen activating proteins (FAPs) described by Szent-Gyorgyi et al., Fluorogen-activating single-chain antibodies for imaging cell surface proteins, Nature biotechnology, 2008) and U.S. Patent Application Publication US-2011-0159519-A1 were not fusions to natural proteins; rather they consisted of a FAP moiety anchored at the cell surface by a minimal transmembrane domain. The FAP system consists of a genetically encodable engineered antibody fragment (scFv) that binds a small organic dye called a fluorogen. The fluorogen is non-fluorescent in solution, but upon binding to the FAP, its fluorescence activity is increased dramatically (up to 15,000-20,000 fold).

To demonstrate the utility of the subject invention, descriptions are provided of the addition of FAP reporters to the termini of a number of exemplary proteins with relevance to human diseases, such as the beta₂ adrenergic receptor (B₂AR), the insulin-regulated glucose transporter (GLUT4), and the cystic fibrosis transmembrane conductance regulator (CFTR). FAP-tagged membrane proteins were visualized selectively at the surface using cell impermeable fluorogens, or throughout the endomembrane system using cell permeable fluorogen. The utility of the invention for monitoring dynamic processes such as membrane protein trafficking was further demonstrated by the observation of corrector-response for reporter-tagged CFTR. Utility is also demonstrated through the detection of internalization of B₂AR with a FAP reporter appended to its C-terminus according to our invention.

Modification of a membrane-protein terminus as described herein has not heretofore been reported nor is there a specific report of a natural example of a member of a membrane protein family that has an extra transmembrane domain at its terminus, for example a 13-pass membrane protein that is a member of a 12-pass membrane protein family. If such examples exist they are not generally recognized, even by those skilled in the art. Nor has the utility of membrane protein constructs with their termini fused to reporter polypeptides and membrane-spanning linkers according to the present invention heretofore been recognized, either in nature or in the literature of recombinant technology. Indeed, the absence of natural and man-made examples would lead one to expect that those proteins that are modified according to the invention described herein would be compromised with respect to structure or function. Contrary to this expectation, the description and examples that follow demonstrate, that proteins modified according to the present invention are not significantly compromised with respect to structure or function.

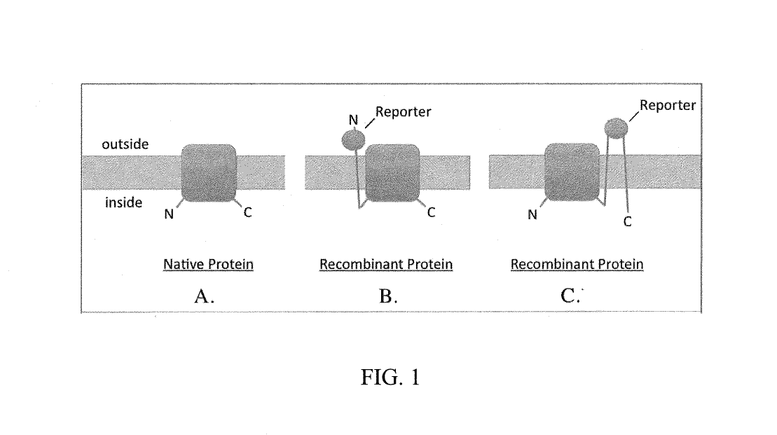

Referring to FIG. 1, embodiments of the recombinant construct (B and C) are shown for a membrane protein (A) having a cytosolic N-terminus (“inside”) and a cytosolic C-terminus (“inside”). The membrane protein may be any membrane protein of interest. Examples include those for which experiments are described herein, such as the beta₂ adrenergic receptor (B₂AR), the insulin-regulated glucose transporter (GLUT4), and the cystic fibrosis transmembrane conductance regulator (CFTR). Other membrane proteins are described in for example, Almen et al., Mapping the human membrane proteome: a majority of the human membrane proteins can be classified according to function and evolutionary origin, BMC biology, 2009.

Those skilled in the art will appreciate that any membrane protein, including proteins that reside in the membranes of cellular organelles, can be recombinantly linked at either its N- or C-terminus to an amino acid sequence that forms the recombinant construct of the present invention.

The membrane spanning linker may be any known transmembrane domain, and more specifically, any membrane spanning amino acid sequence that can be recombinantly linked to a terminus of a membrane protein. The linker in the exemplary embodiments described here is the membrane spanning domain from the human platelet derived growth factor receptor (PDGFR). Many thousands of other membrane spanning domains are known and would be familiar and available to one skilled in the art for incorporation into proteins modified according to the invention. (Hubert et al., Single-spanning transmembrane domains in cell growth and cell-cell interactions: More than meets the eye?, Cell adhesion & migration, 2010.)

The membrane spanning linker may comprise a polypeptide sequence having at least about 85%, at least about 90%, at least about 95%, about 96%, about 97%, about 98%, about 99% or about 100% sequence identity to the polypeptide sequence of the membrane spanning domain of human platelet derived growth factor receptor (PDGFR), (SEQ ID NO.: 17), or to a polypeptide sequence selected from the group consisting of the transmembrane domains from CD4TL, (GenBank Accession: NM_{—}000616.4) for which the method of constructing the domain is disclosed in Barriere et al., Molecular basis of oligoubiquitin-dependent internalization of membrane proteins in Mammalian cells, Traffic, 2006, incorporated herein by reference, EGFR (GenBank Accession: NM_{—}005228), VEGFR1 (GenBank Accession: NM_{—}001159920.1), VGFR2 (GenBank Accession: NM_{—}002253), VGFR3 (GenBank Accession: NM_{—}182925.4), FGFR1-4 (GenBank Accession: NM_{—}001079908.1, NM_{—}000141.4, NM_{—}000142.4, and NM_{—}008011.2), FGFR6 (NCBI Gene ID: 2265) and TGFBR1-3 (GenBank Accession: NM_{—}001130916.1, NM_{—}001024847.2, and NM_{—}003243.4.), each of the foregoing incorporated herein by reference.

The properties and sequences of many thousands of natural or synthetic transmembrane domains, generally comprising a sequence of about twenty nonpolar amino acids that forms a thermodynamically stable alpha-helix in the membrane, are known and too numerous to list herein. In light of the disclosures provided herein, those skilled in the art will recognize that any such transmembrane domain may be recombinantly expressed with a membrane protein of interest and a reporter polypeptide, such as a FAP reporter polypeptide, to form the recombinant protein construct of the present invention.

The reporter polypeptide may be any polypeptide with properties, such as fluorescence, luminescence, fluorogen activation, specific interaction with a known antibody, enzymatic activity for a chromogenic substrate, or covalent receptor activity for a small molecule such as biotin or alkyl halides, that enable specific detection of the reporter in tissues, cells, or biochemical preparations derived therefrom. Numerous reporter polypeptides are known to one skilled in the art, Jiang et al., Recent developments of biological reporter technology for detecting gene expression, Biotechnology & genetic engineering reviews, 2008; Mehta and Zhang, Reporting from the field: genetically encoded fluorescent reporters uncover signaling dynamics in living biological systems, Annual review of biochemistry, 2011. Exemplary reporters include enzymes such as beta-galactosidase or alkaline phosphatase that can be detected using chromogenic or fluorogenic substrates, fluorescent proteins such as GFP or RFP, luminescent proteins such as firefly or Renilla luciferase, epitope tags such as cMyc or FLAG or 6× His, biotinylation acceptor sequences such as AviTag, proteins that bind small molecules covalently such as SNAP-tag and HALO-tag or non-covalently such as glutathione S-transferase, and fluorogen-activating proteins (FAPs), such as MG13 (also sometimes referred to as HL4-MG in the literature), dNP-138 (also referred to as L5-MG dimer) m scFv1 (sometimes referred to as HL1-TO1 in the literature), or AM2.2 (also sometimes referred to as HL1.0.1-TO1 in the literature).

In certain embodiments the reporter may be one of the following exemplary single chain or dimerized FAPs: HL1-TO1, HL1.1-TO1, HL1.0.1-TO1, HL4-MG, L5-MG, H6-MG, K7, M8, dNP138 (SEQ. ID. NOS.: 33, 34, 26, 35, 36, 37, 38, 39, and 40, respectively) and others described in Ozhalici-Unal et al., A rainbow of fluoromodules: a promiscuous scFv protein binds to and activates a diverse set of fluorogenic cyanine dyes, Journal of the American Chemical Society, 2008; Szent-Gyorgyi et al., Fluorogen-activating single-chain antibodies for imaging cell surface proteins, Nature biotechnology, 2008; Falco et al., scFv-based fluorogen activating proteins and variable domain inhibitors as fluorescent biosensor platforms, Biotechnology journal, 2009; Fitzpatrick et al., STED nanoscopy in living cells using Fluorogen Activating Proteins, Bioconjugate chemistry, 2009; Fisher et al., Detection and quantification of beta2AR internalization in living cells using FAP-based biosensor technology, Journal of biomolecular screening, 2010; Holleran et al., Fluorogen-activating proteins as biosensors of cell-surface proteins in living cells, Cytometry. Part A: the journal of the International Society for Analytical Cytology, 2010; Zanotti et al., Blue fluorescent-dye protein complexes based on fluorogenic cyanine dyes and single chain antibody fragments, Organic & biomolecular chemistry, 2011; Holleran et al., Pharmacological Rescue of Mutant CFTR Detected Using a Novel Fluorescence Platform, Molecular medicine, 2012; Saunders et al., Fluorogen activating proteins in flow cytometry for the study of surface molecules and receptors, Methods, 2012; Senutovitch et al., A variable light domain fluorogen activating protein homodimerizes to activate dimethylindole red, Biochemistry, 2012. Each of the foregoing is hereby incorporated herein by reference.

The DNA sequences that encode for the amino acid sequences of several FAP reporter polypeptides are provided in Szent-Gyorgyi et al., Fluorogen-activating single-chain antibodies for imaging cell surface proteins, Nature biotechnology, 2008. The DNA sequences may function as vectors in a process for preparing the recombinant proteins described herein. The reporter polypeptide may comprise a polypeptide sequence having at least about 85%, at least about 90%, at least about 95%, about 96%, about 97%, about 98%, about 99% or about 100% sequence identity to the polypeptide sequences of FAPs: HL1-TO1, HL1.1-TO1, HL1.0.1-TO1, HL4-MG, L5-MG, H6-MG, K7, M8, dNP138 (SEQ. ID. NOS.: 33, 34, 26, 35, 36, 37, 38, 39 and 40, respectively), or of other reporter polypeptides.

Exemplary fluorogens that would be used to bind to cognate recombinant protein constructs of the present invention include malachite green and analogs and derivatives thereof, thiazole orange and analogs and derivatives thereof, and any of the cyanine or oxazole thiazole blue fluorogens disclose in K. J. Zanotti, et al., Blue Fluorescent Dye-Protein Complexes Based on Fluorogenic Cyanine Dyes and Single Chain Antibody Fragments, Org. bio. chem. 2011 Feb. 21; 9(4) 1012-1020; Representative thiazole orange (TO) fluorogens include TO1-2p, and TO-11p. Representative malachite green (MG) fluorogens include MG-11P, MG-ester, and MG-2p.

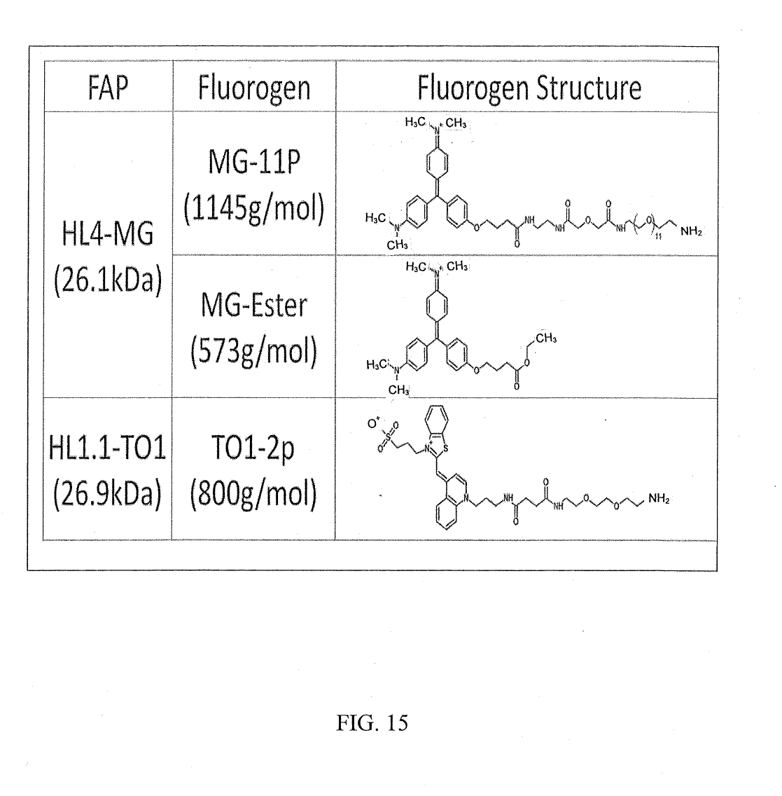

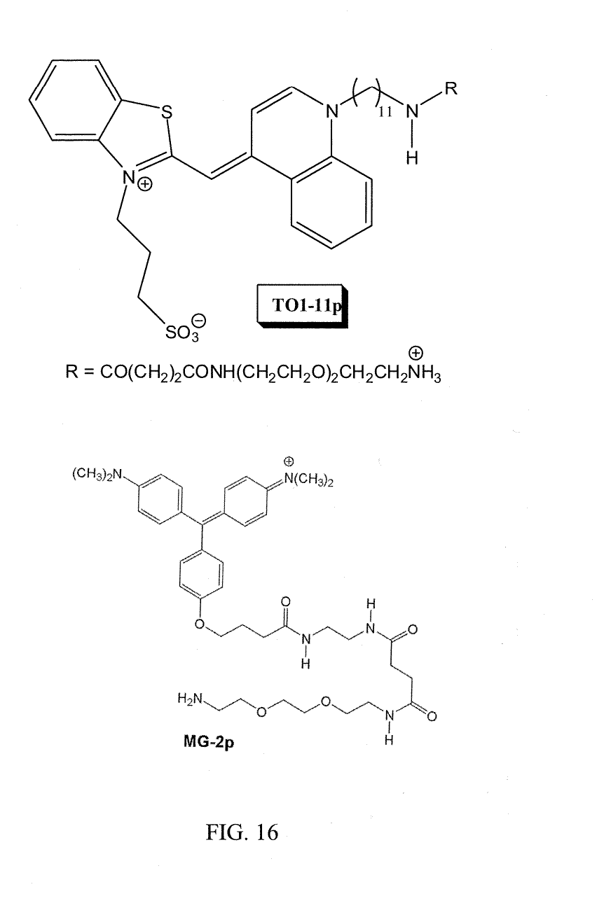

Properties and structures for three representative fluorogens, MG-11P, MG-ester and TO1-2P, that selectively bind to two different FAPs, HL4-MG and HL1.1-TO1, respectively, are shown in FIG. 15. Structures for additional fluorgens are shown in FIG. 16.

### EXPERIMENTAL

Materials and Methods

Reagents:

(2)-Isoproterenol(1)bitartrate and DL-Propranolol HCL were from Sigma-Aldrich (St. Louis, Mo.). Cellstripper was purchased from Mediatech (Manassas, Va.).

Plasmids and Retroviral Vectors:

### EXAMPLE 1

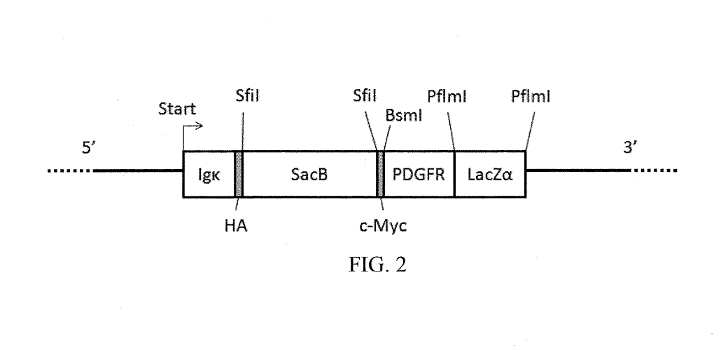

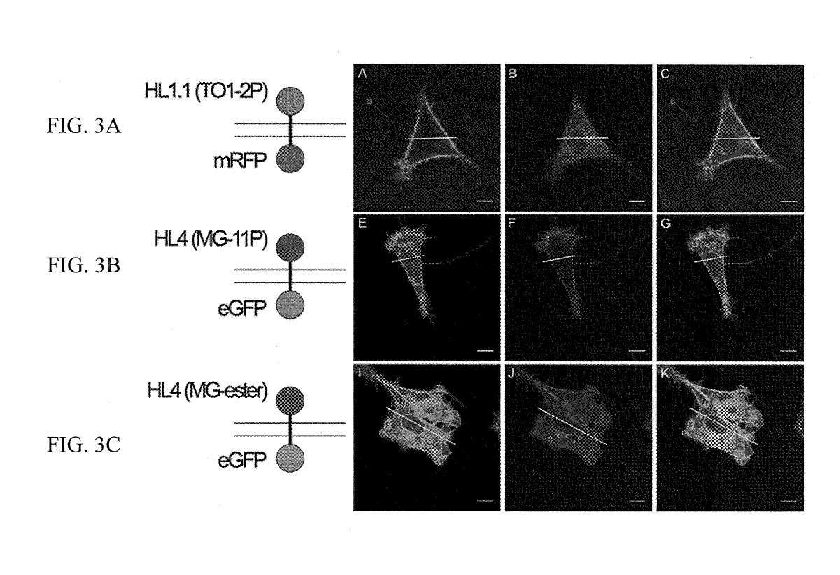

Expression vector pDisplaySacLac2, shown in FIG. 2, was constructed as follows. The SacB gene was PCR-amplified from the vector pDNR-1r (obtained from Clontech, Mountain View Calif.) using primers TATATAGGCCCAGCCGGCCCCACATATACCTGCCGTTCAC (SEQ. ID. NO.: 1) and TATATAGGCCCCTGCGGCCACGTCAATGCCAATAGGATATCG (SEQ. ID. NO.: 2). This amplicon was cut with Sfil and cloned into Sfil-digested pDisplayBlue (see, Szent Gyorgyi et al., Nature Biotechnology, 26:235-240 (2008)) to produce pDisplaySac. Primers AGAGGATCTGAATGCTGTGG (SEQ. ID. NO.: 3) and CTCGAGCTAACGCCACCTGCTGGCATCGTCCAGGCTGTGGACGTGGCTTCTTCT GCCAA (SEQ. ID. NO.: 4) were used to generate an amplicon from a pDisplayBlue template that contained the platelet derived growth factor receptor (PDGFR) transmembrane-domain; the amplicon was cut with Bsml and Xhol and ligated into Bsml/Xhol-digested pDisplaySac. The resulting construct was digested with PflMI. Primers CCACAGCCTGGGTTAGCTCACTCATTAGGCA (SEQ. ID. NO.: 5) and CTCGAGCTAACGCCACCTGCTGGCATCGTCCAGGCTGTGGACGTGGCTTCTTCT GCCAA (SEQ. ID. NO.:6) were used to generate an amplicon from a pDisplayBlue template that contained the platelet derived growth factor receptor (PDGFR) transmembrane-domain; the amplicon was cut with Bsml and Xhol and ligated into Bsml/Xhol-digested pDisplaySac. The resulting construct was digested with PflMI. Primers CCACAGCCTGGGTTAGCTCACTCATTAGGCA (SEQ. ID. NO.: 7) and CCACCTGCTGGCTAACGCCAGTTTGAGGGGACGACGA (SEQ. ID. NO.: 8) were used to generate an amplicon from a pDisplayBlue template that contained the lacZa-complementing fragment, and PflMI-digested amplicon was ligated into the construct to create pDisplaySacLac2.

### EXAMPLE 2

Retroviral expression vector pBabeSacLac2 was constructed as follows. pBabe-puro-H-Ras-V12 (obtained from Addgene, Cambridge Mass.), which carries an Sfil site upstream of the puro-mycin resistance gene, was linearized with Sfil, treated with T4 polymerase to create blunt ends, and self-ligated to create a derivative without an Sfil site. This plasmid was digested with BamHl and SalI to produce a recipient fragment. pDisplaySacLac2 was digested with BamHl and Xhol, and a 650 bp BamHll/Xhol fragment was recovered and ligated into the recipient fragment to produce pBabeSacLac2.

Generation of Vector Inserts:

Sfil sites were added to the ends of open reading frames by PCR. 5′ primers added the 13-nt sequence GGCCCAGCCGGCC (SEQ. ID. NO.: 9) to the extreme 5′ end of the open reading frame (ORF), and 3′ primers added the 13-nt sequence GGCCGCAGGGGCC (SEQ. ID. NO.: 10) to the extreme 3′ end of the open reading frame. Amplicons were cloned into the vectors pCR2.1-TOPO or pCR-Blunt II-TOPO (obtained from Invitrogen, Carlsbad Calif.), sequenced to confirm that no mutations were introduced during amplification, and removed from the vector by SfiI digestion. Molecules were generated in this way for six open reading frames; EGFP (template: pStealth, (Telmer C. A., et al., Epitope tagging genomic DNA using a CD-tagging Tn10 minitranspson, Biotechniques, 32:422-424 (2002), incorporated herein by reference), mRFP (template: pACT-mRFP-Mem, gift of Dr. Y. Saeki, Ohio State University, incorporated herein by reference), HL1.1-TO1 (template: pNL6-HL1.1-TO19 (see, Szent Gyorgyi et al., Nature Biotechnology, 26:235-240 (2008)), HL4-MG [template pNL6-HL4-MG, (see, Szent Gyorgyi et al., Nature biotechnology, 2008, supra.)], human GLUT4 (template: cDNA clone 726246, obtained from Open Biosystems, Huntsville, Ala., Genbank accession number: BC069621, incorporated herein by reference), and human CFTR (Genbank accession number: NM_{—}000492, incorporated herein by reference). These two amplicons also included the template stop codon; in the other cases a stop codon was not included. A seventh fragment containing a complete open reading frame and stop codon from the ADRB2 gene, encoding the B₂AR receptor (GenBank accession: AAF20199.1, incorporated herein by reference; template: fosmid Wl2-2202O9/G248P86156H5, obtained from BACPAC Resources Center, Oakland Calif.), was prepared in an equivalent manner but with the sequence flanked by BsmI sites.

Cell Lines:

NIH 3T3 cells were obtained from the American Type Culture Collection (Manassas, Va., ATCC Accession No. CRL-1658). C2C12 cells, ATCC Accession No. CRL-1772, were the gift of Dr. P. Campbell, Carnegie Mellon University. HEK 293 cells ATCC Accession No. CRL-1573, were the gift of Dr. R. Frizzell, University of Pittsburgh School of Medicine.

Transfection:

Transfections were performed using Minis TransIT1-LT1 Transfection Reagent (obtained from Minis, Madison Wis.) and pDisplaySacLac (see, Swift, S., et al., Rapid production of retroviruses for efficient gene delivery to mammalian cells using 293T cell-based systems, Current Protocols in Immunology, Unit 10.28, Suppl. 31 (1999)) vectors. For 35 mm dishes: DNA (2.5 IL, concentration of 1 lg/mL) was added to 7.5 IL Trans IT1-LT1 in 250 I L of DMEM. Transfection complexes were allowed to form for 30 min and subsequently added to cells grown in antibiotic free DMEM with 10% calf serum. Transfection complexes were removed and medium changed after 24 h. After 2 or more weeks of growth, stable transfectants were obtained by FACS as described previously (see, Szent Gyorgyi et al, Nature biotechnology, 2008, supra.).

Transduction:

Transducing particles for pBabeSacLac2 constructs were generated using the Phoenix Ecotropic Packaging System (Nolan laboratory, Stanford University). Phoenix-Ecotropic cells were plated at 1.3×10⁶ cells/75 cm (Swift, S., et al., Current Protocols in Immunology, Unit 10.28, Suppl. 31 (1999)) flask in DMEM with calf serum without antibiotics. pBabeSacLac2 DNA was transfected as described above scaled to surface area. After 24 h, transfection complexes were removed and replaced with 8 mL of DMEM with calf serum and incubated for 48 h at 328° C./5% CO₂. Medium was removed and filtered through Millex-HV 0.45 I m syringe filter and flash frozen in liquid nitrogen. Recipient cells were plated at 2×10⁵ cells/35 mm dish 24 h before transduction. Cells were infected by adding viral supernatant and 6 Ig/mL of hexadimethrine bromide and incubated for 24 h at 328° C./5% CO₂. Cells were replated in 75 cm (Swift, S., Current Protocols in Immunology, Unit 10.28, Suppl. 31 (1999)., supra.) flasks and screened for expression 48 h later.

Fluorescence Microscopy:

Cells were grown in DMEM plus 10% calf serum in 23 mm glass-bottom dishes (obtained from Mattek, part no p35G-1.5-14-C) and imaged with Carl Zeiss LSM 510 Meta/UV DuoScan Inverted Spectral Confocal Microscope at 63× objective magnification. For cells expressing FAP HL1.1-TO1, 40 nM TO1-2P, a membrane-impermeant fluorogen, was added 5 min. before imaging. For cells expressing HL4-MG, 40 nM MG-11P, a membrane-impermeant fluorogen, or 40 nM MG-ester, a membrane-permeant fluorogen, were added 10 min. before imaging. Times (5 min. and 10 min.) were shown in pilot experiments to be sufficient to reach saturation with regard to signal intensity and location. Excitation and emission wavelengths were, respectively, 488 nm and 505-550 nm for EGFP, 561 nm and 575-615 nm for mRFP, 488 nm and 505-550 nm for HL1.1-TO1, and 633 nm and LP 650 nm for HL4-MG. Illumination intensity and detector gain settings on the microscope were held constant for all observations.

B₂AR Internalization:

Imaging was performed using an Olympus IX50 microscope equipped with a spinning disk confocal imaging system (Solamere Technology Group, Salt Lake City, Utah). For TO1-2P, excitation was a 488 Argon laser and a 500 nm long pass filter for emission (HQ500LP; Chroma Technologies). NIH 3T3 cells expressing HL1.1-TO1-B₂AR were incubated in the presence of 100 nM TO1-2P and stimulated with 10 I M isoproterenol or untreated (control) for 40 min, followed by confocal image acquisition at 378° C. with 10% CO₂.

Expression of Membrane-Spanning Linkers

Proteins with FAP and FP Domains

To facilitate the expression of membrane-associated proteins carrying FAP or fluorescent protein domains, two new expression vectors were constructed, the plasmid vector pDisplay SacLac2 and the retroviral vector pBabeSacLac2. In pDisplaySacLac2 transgene expression is driven by the CMV immediate early promoter. In pBabeSacLac2 expression is driven by the MMLV LTR promoter. Both vectors express a translation unit that includes (from 5′ to 3′) a leader sequence derived from a murine lgκ gene (SEQ. ID. NO.: 15), an HA epitope (SEQ. ID. NO.: 28), a SacB stuffer flanked by Sfil restriction sites, a c-Myc epitope, a Bsml restriction site, a transmembrane domain taken from the human platelet derived growth factor receptor B gene (PDGFRB) (SEQ. ID. NO.: 17) (NCBI Reference Sequence: NM 002609.3; Gronwald et al., Cloning and expression of a cDNA coding for the human platelet-derived growth factor receptor: evidence for more than one receptor class, Proceedings of the National Academy of Sciences of the United States of America, 1988, each incorporated herein by reference), and a LacZa stuffer flanked by PflMI restriction sites. Cleavage of the vector with Sfil or PflMI releases the respective stuffer sequences and leaves the same noncomplementary 3′ three-base overhangs. Thus, if one prepares an open reading frame of interest with appropriate 3-base overhangs, it can be readily cloned between either the Sfil or the PflMI sites in a directed fashion (Telmer C A, et al., Detection and assignment of mutations and minihaplotypes in human DNA using peptide mass signatures genotyping (PMSG): Application to the human RDS/peripherin gene, Genome Res., 13:1944-1951(2003)). A schematic view of the relevant translation unit in the vectors is shown in FIGS. 3A-3C.

The PDGFRB transmembrane domain inserted in the example above may be substituted by nucleotide sequences encoding for any desired transmembrane polypeptide or fragment thereof, such as, but not limited to, membrane spanning linkers selected from PDGFR, CD4TL, EGFR, VEGFR1, VGFR2, VGFR3, FGFR1-4, FGFR6 and TGFBR1-3.

Open reading frames encoding the FAP's HL1.1-TO1 and HL4-MG, were cloned between the Sfil or PflMI sites in pDisplaySacLac2 or pBabeSacLac2 to give constructs that express recombinant single-pass transmembrane proteins with their N-terminal domains outside the cell and their C-terminal domains inside the cell. NIH 3T3 cells that stably express the constructs were isolated and examined by fluorescence microscopy after fluorogen addition, if appropriate.

Images of cells expressing three different recombinant protein constructs are shown in FIGS. 3A-3C. FIGS. 3A and 3B are FAP-FP expressing cells in the presence of impermeable fluorogen. Image analysis was performed using the Image J software by specifying a linear region of interest and using the plot profile operation. FIG. 3A represents the topology of an embodiment of fusion protein HL1.1(TO1 2P) and fluorescence channels A, B, and C in different colors (e.g., green (A), red (B), and a merger of A and B (C)). FIG. 3B represents the topology of fusion protein HL4(MG 11P) and fluorescence channels E, F, and G in different colors (e.g., green (E), red (F), and a merger of E and F (G)). When visualized with the membrane-impermeant fluorogens shown in FIG. 15, both the FAP-mRFP (image A in FIG. 3A) and FAP-EGFP (image F in FIG. 3B) constructs showed signal exclusively at the cell surface. RFP and GFP signal was also present at the cell surface, but in marked contrast to the FAP patterns, much of the signal came from inside the cell.

FIG. 3C represents the topology of fusion protein HL4(MG ester) and fluorescence channels I, J, and K in different colors (e.g., green (I), red (J), and a merger of I and J (K)). FIG. 3C represent cell permeable fluorogen labeling of FAP-FP cells. Extensive internal signal was also observed for cells expressing the HL4-MG-EGFP construct (image J in FIG. 3C) that were incubated with membrane-permeant fluorogen.

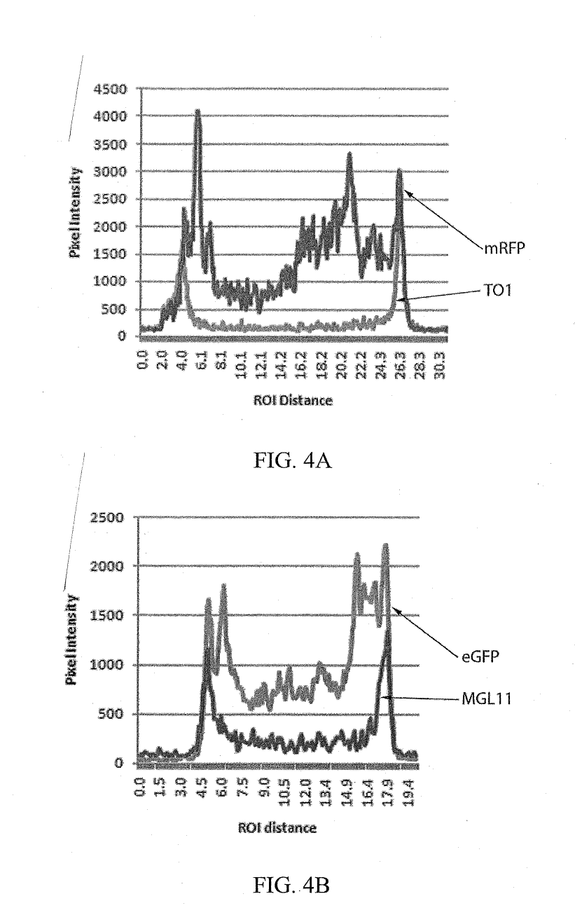

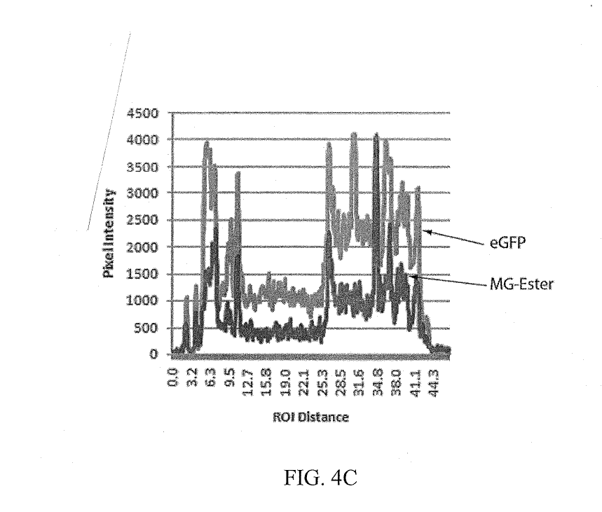

Histograms of the fluorescent images of FIG. 3A-C, respectively, are shown in FIGS. 4A-C. FIG. 4A shows a histogram generated from the fluorescence images of FIG. 3A (A-C) by plotting the pixel intensity across a specified region (indicated by the white line in the fluorescence images). FIG. 4B shows a histogram generated from the fluorescence images FIG. 3B (E-G) by plotting the pixel intensity across a specified region (indicated by the white line in the fluorescence images). FIG. 4C shows a histogram generated from the fluorescence images 3C (LK) by plotting the pixel intensity across a specified region (indicated by the white line in the fluorescence images).

The fluorogen activating proteins used in the examples above may be substituted by any other desired fusion partner in the manner described. Exemplary substitute FAPs selected from the group consisting of HL1-TO1, HL1.1-TO1, HL1.0.1-TO1, HL4-MG, L5-MG, H6-MG, K7, M8 and dNP138 or any other FAP or reporter polypeptide identified herein or encompassed by the present invention may be substituted using known techniques.

Expression of Human Membrane Proteins with N-Terminal FAP-Tags

Beta2 Adrenergic Receptor:

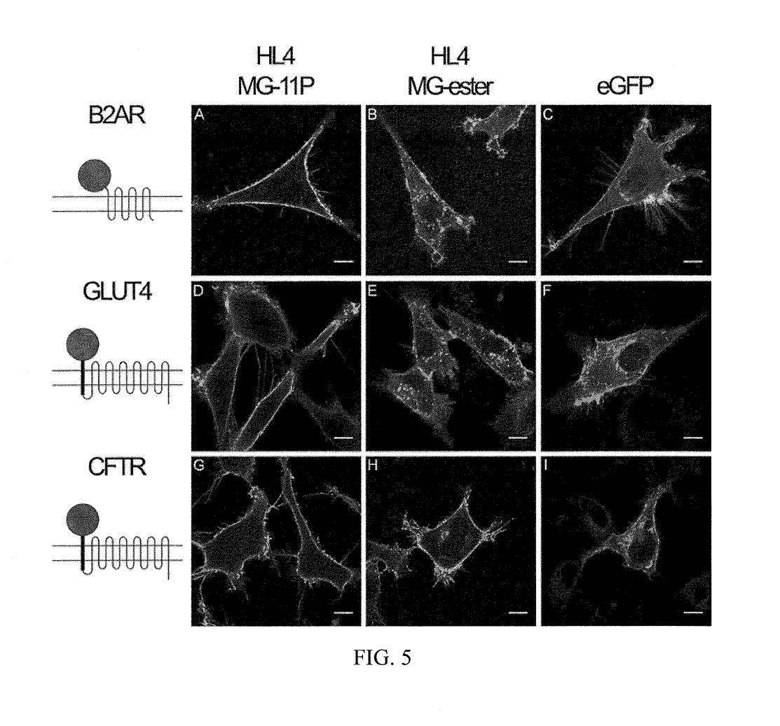

The human b-2 adrenergic receptor (B₂AR, encoded by the gene ADRB2, GenBank accession number: AAF20199.1, incorporated herein by reference) (template: fosmid WI2-2202O9/G248P86156H5, BACPAC Resources Center, Oakland Calif., incorporated herein by reference) is a GPCR with an extracellular N-terminus, seven transmembrane domains, and a cytoplasmic C-terminus. B₂AR responds to the hormones epinephrine and norepinephrine as well as a large number of known agonists and antagonists, including several important drugs used to treat asthma and other respiratory conditions (Moore et al., Long-acting inhaled beta2-agonists in asthma therapy, Chest, 1998). To create vectors that express B₂AR with FAP or fusion protein (FP) domains at their N-termini, an open reading frame encoding human B₂AR was ligated into the Bsml site of pBabeSacLac2, and open reading frames encoding HL1.1-TO1, HL4-MG, or EGFP were ligated between the Sfil sites in place of the SacB stuffer. Fluorescence micrographs of cells expressing B₂AR fusion proteins are shown in FIGS. 5A-5C. When imaged after exposure to nonpermanent fluorogen, HL4-MG-B₂AR cells showed signal exclusively at the cell surface (FIG. 5A). In contrast, cells expressing HL4-MG-B₂AR that were exposed to membrane-permeant fluorogen (FIG. 5B), and cells expressing EGFP-B₂AR protein, (FIG. 5C) showed extensive internal signal as well as surface labeling.

Glucose Transporter 4.

Human glucose transporter 4 (GLUT4, encoded by the SLC2A4 gene, SEQ. ID. NO.: 21) is a type II 12-pass transmembrane protein. GLUT4 is the primary insulin-responsive glucose transporter in muscle and fat cells and plays a central role in the pathology of Type 1 and Type 2 diabetes Hou and Pessin, Ins (endocytosis) and outs (exocytosis) of GLUT4 trafficking, Current opinion in cell biology, 2007. To create vectors that express recombinant GLUT4 with extracellular N-terminal FAP or FP domains, an open reading frame encoding mature human GLUT4 (SEQ. ID. NO.: 21) (NCBI Reference sequence: BC069615.1, incorporated herein by reference) was ligated between the PflMI sites of pBabeSacLac2, and open reading frames encoding HL1.1-TO1 (SEQ. ID. No.: 20), HL4-MG (SEQ. ID. NO.: 35), or EGFP were ligated between the Sfil sites. The nucleotide sequence for TO1.1-GLUT4 is shown in SEQ. ID NO.: 19. The proteins expressed from these recombinant genes were expected to have 13 transmembrane domains with the FAP or FP exposed extracellularly. Fluorescence micrographs of C2C12 cells expressing GLUT4 constructs are shown in FIG. 5B. When imaged after exposure to non-permeant fluorogen, FAP-GLUT4 cells showed signal exclusively at the cell surface. (See FIG. 5B, image D) Like the B₂AR expressing cells, FAP-GLUT4 expressing cells exposed to membrane-permeant fluorogen (FIG. 5B, image E), and EGFP-GLUT4 cells (FIG. 5B, image F) showed extensive internal signal as well as surface labeling.

Cystic Fibrosis Transmembrane Conductance Regulator.

The cystic fibrosis transmembrane conductance regulator (encoded by the CFTR gene, (SEQ. ID. NO. 18)) is a type II 12-pass membrane protein of the ABC transporter class. Mutations in CFTR are responsible for the human genetic disease, cystic fibrosis (Ameen N, et al., Endocytic trafficking of CFTR in health and disease, J. Cyst. Fibros., 6:1-14 (2007)). To create vectors that express CFTR with extracellular FAP or FP domains at their N-termini, an open reading frame encoding mature human CFTR (NCBI Reference Sequence: NM_{—}000492.3, incorporated herein by reference) was ligated between the PflMI sites of pBabeSacLac2, and open reading frames encoding HL1.1-TO1, HL4-MG, or EGFP were ligated between the Sfil sites. These vector constructs were expected to express proteins with topologies identical to those of the GLUT4 constructs. Fluorescence micrographs of HEK 293 cells expressing FAP or EGFP CFTR constructs are shown in FIG. 5C. When imaged after exposure to nonpermeant fluorogen, the FAP construct showed signal exclusively at the cell surface (FIG. 5C, image G). As with the B₂AR and GLUT4 expressing cells, FAP-CFTR expressing cells exposed to membrane-permeant fluorogen (FIG. 5C, image H) showed internal signal as well as surface labeling, just as did cells expressing EGFP-CFTR (FIG. 5C, image I).

The membrane proteins used in the examples above may be substituted for any other desired membrane protein or desired fragment thereof in the manner described or using other methods and procedures well known in the art.

B₂AR Internalization in Response to Agonist.

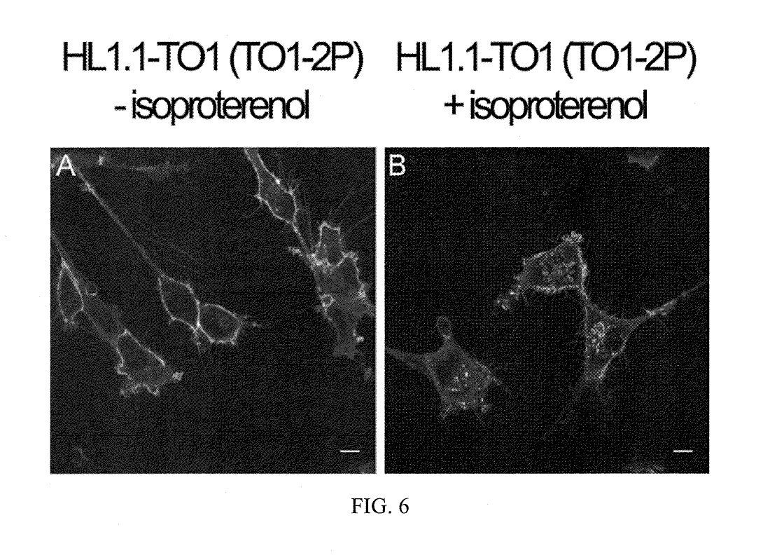

For many G-protein coupled receptors (GPCRs) including B₂AR, stimulation with agonist leads to G-protein activation in seconds followed by internalization of the receptor over the following minutes through a process known as receptor downregulation (McLean A. J., et al., Ligand regulation of green fluorescent protein-tagged forms of the human beta(1)- and beta(2)-adrenoceptors: comparisons with the unmodified receptors, Br. J. Pharmacol., 130:1825-1832 (2000)). As showing in FIGS. 6A and B, treatment of FAP-tagged B₂AR with a standard dose of the agonist isoproterenol in the presence of membrane-impermeant fluorogen led to signal internalization, indicating that FAP-tagged proteins can be used in concert with membrane-impermeant fluorogens to monitor membrane protein trafficking in living cells. FIG. 6A represents cells expressing HL1.1-B₂AR that were incubated in 100 nM TO1-2P, a membrane impermeant fluorogen, for 40 min., then imaged by confocal microscopy. FIG. 6B represents cells expressing HL1.1-B₂AR that were incubated in 100 nM TO1-2P, a membrane impermeant fluorogen plus 10 lM of the agonist isoproterenol for 40 min., then images by confocal microscopy.

FAP Reporter Polypeptides Enable Selective Visualization of Surface-Exposed Protein

The need to precisely identify the subcellular location of proteins is increasingly important in fluorescence imaging. Chemical labeling of genetically encoded tags with small-molecules allows for spatial and temporal control over protein visualization with the ability to utilize chemically diverse and environmentally sensitive probes. Techniques for selective chemical labeling of cell surface proteins, ACP-PPTase and biotin-ligase based methods, had heretofore proven useful; however, these labels are irreversible and require multiple wash steps with prolonged incubation times (Chen I, et al., Site-specific labeling of cell surface proteins with biophysical probes using biotin ligase, Nat Methods, 2:99-104 (2005); George N, et al., Specific labeling of cell surface proteins with chemically diverse compounds, J. Am. Chem. Soc., 126:8896-8897 (2004)). FAP-based imaging combines the ability to visualize dynamic biological processes with the specificity of chemical labeling methods.

Each of the expressed FAP-tagged proteins was visualized exclusively at the cell surface when cultures were exposed to membrane-impermeant fluorogen. Signal appeared inside the cell only when tagged protein bound to fluorogen was internalized by endocytosis (FIG. 6). In contrast, when cells expressing FAP-tagged membrane proteins were exposed to membrane-permeant fluorogen (MG-ester, FIG. 15), fluorescence was observed not just at the cell surface but also in the nuclear membrane, the Golgi, and in vesicles of various sizes. Given that the nuclear membrane is contiguous with the membrane of the endoplasmic reticulum, it is believed that the signal observed at the nuclear membrane represents fusion-protein molecules that have arrived there by lateral diffusion from the endoplasmic reticulum membrane. Signal in the Golgi is believed to represent proteins passing through that organelle on their way to the cell surface. The punctuate signal seen throughout the cytoplasm is believed to represent exocytic and/or endocytic vesicles carrying fusion proteins to or from the plasma membrane.

The orientation of the fusion proteins used in the foregoing examples is such that the FAP domain projects into the lumen of any intracellular membrane-bounded compartment that it may occupy. Thus, MG-ester molecules would need to transit both the plasma membrane and the organelle membrane to reach the FAP and yield fluorescent signal. The observation of such signal implies that after entering the cytoplasm, MG-ester molecules are not immediately converted to charged membrane-impermeable molecules by the action of cellular esterases. The results with the HL4-EGFP recombinant protein constructs provide particularly strong support for this interpretation in that the recombinant protein shows essentially the same location pattern whether visualized with MG-ester or by EGFP fluorescence (FIG. 3C, image K).

It has also been shown that the SacLac modular cloning system facilitates generation of FAP tagged fusion proteins. B₂AR, GLUT4, and CFTR have been successfully tagged and demonstrated that they can be detected at the cell surface. Furthermore, the dynamic trafficking of proteins in living cells was shown by agonist induced endocytosis of HL1.1-TO1-B₂AR. These FAP fusion proteins provide a platform for examining trafficking properties specific to each protein.

In one aspect of the invention, the recombinant constructs may be used in assays that detect translocation of proteins to or from the cell surface in response to specific stimuli. For example, to assay for protein internalization, cells may be incubated in membrane-impermeant fluorogen before and after stimulation. Examination of the cells for a reduction in fluorescent signal consequent to stimulation would follow. In another aspect of the invention, the recombinant protein constructs may be used to assay for protein delivery to the plasma membrane. Cells may be incubated in membrane-impermeable fluorogen and examined, for example, by fluorescent microscopy, for an increase in signal consequent to stimulation. In each aspect, examination and collection of data may be by fluorescence microscopy, or by higher throughput means such as flow cytometry or fluorimetry in a microwell format. Those skilled in the art will recognize that other suitable known means of examining the labeled cells may be used to detect a signal. While detection by use of fluorescent signals have been described, other signal detection means may be used. Examples include high content imaging systems, image based flow cytometry and spectroscopy.

The FAP-Cystic Fibrosis Transmembrane Conductance Regulator (CFTR) Platform:

Many small molecule compounds called correctors have been identified that partially rescue the trafficking defect of F508de1-CFTR, but problems with efficacy and reliability, reduced throughput for drug screening and the introduction of artifacts due to cell permeabilization have been identified. Selective and sensitive detection of F508de1-CFTR at the cell surface can be used to identify new correctors and can elucidate mechanisms for existing ones. However, this approach normally requires labor intensive biochemical methods, such as immunofluorescence using epitope tags or biotinylation and subsequent immunoblotting techniques. Okiyoneda et al., Peripheral protein quality control removes unfolded CFTR from the plasma membrane. Science, 2010; Luo, et al., Trafficking of immature DeltaF508-CFTR to the plasma membrane and its detection by biotinylation, The Biochemical Journal, 2009; Gentzsch et al., Endocytic trafficking routes of wild type and DeltaF508 cystic fibrosis transmembrane conductance regulator, Molecular biology of the cell, 2004. To address these limitations, CFTR was tagged with an embodiment of the recombinant protein construct of the present invention. In various embodiments, the construct is comprised of a genetically encoded reporter polypeptide, such as a FAP, recombinantly linked, or fused, through a membrane spanning linker to the N-terminus of the CFTR protein. The construct provides a unique and selective fluorescence assay of the abundance of this protein at the cell surface.

The FAP-CFTR platform, whose behavior is consistent with that of untagged wild type (WT) and F508de1-CFTR, retains functional activity. Importantly, F508de1-CFTR FAP constructs provide a method to quantify corrector efficacy that was consistent with functional measurements of endogenous untagged CFTR in a native airway cell background. Using this method, two new correctors, CFFT-002 and C18, were characterized, as well as corrector combinations that significantly improve the trafficking of F508de1-CFTR compared to the previously described C4 corrector. Furthermore, this platform has a modular design to allow for rapid construction of FAP fusions with most transmembrane proteins. Therefore, the recombinant construct described herein can serve as a template for application to other protein trafficking diseases, to accelerate the discovery of pharmacological agents that rescue trafficking defects.

Materials and Methods Plasmids and Constructs

N-Terminal Fusions

FAP fusions to the N terminus of CFTR (SEQ. ID. NO.: 18) with an additional membrane spanning segment (SEQ. ID. NO.: 17) were generated using the pBabeSacLac2 plasmid, described in Example 2. Briefly, CFTR (both wild type (WT) and F508de1) were amplified by PCR from pcDNA3 using primers that provided SfiI restriction sites as follows:

(SEQ. ID. NO.: 11)
Forward: GGCCCAGCCGGCCATGCAGAGGTCGCCTCTGGAA;
(SEQ. ID. NO.: 12)
Reverse: GCCCCTGCGGCCCTAAAGCCTTGTATCTTGCAC.

First, a malachite green (MG) binding FAP (dNP138, nucleotide sequence: TACCCATACGACGTTCCAGACTACGCTCTGCAGGCTAGTGGTGGTGGTGGTTCT GGTGGTGGTGGTTCTGGTGGTGGTGGTTCTGCTAGCCAGGCCGTCGTTACCCAA GAACCTAGTGTTACCGTTAGCCCAGGTGGTACTGTTATACTTACTTGTGGAAGT GGTACGGGTGCCGTCACATCTGGTCATTATGCAAATTGGTTTCAACAAAAACCA GGACAAGCTCCAAGAGCTTTGATTTTTGATACTGATAAGAAGTATTCTTGGACC CCAGGTAGATTTTCTGGATCTTTGCTGGGAGCAAAGGCAGCTTTGACAATATCA GATGCTCAGCCTGAGGACGAAGCCGAGTATTACTGTTCTCTTAGCGACGTGGAT GGCTACTTGTTTGGCGGTGGAACACAACTGACGGTTCTGTCCGGTGGTGGCGGC TCTGGTGGCGGTGGCAGCGGCGGTGGTGGTTCCGGAGGCGGCGGTTCTCAGGC TGTGGTGACTCAGGAGCCGTCAGTGACTGTGTCCCCAGGAGGGACAGTCATTC TCACTTGTGGCTCCGGCACTGGAGCTGTCACCAGTGGTCATTATGCCAACTGGT TCCAGCAGAAGCCTGGCCAAGCCCCCAGGGCACTTATATTTGACACCGACAAG AAGTATTCCTGGACCCCTGGCCGATTCTCAGGCTCCCTCCTTGGGGCCAAGGCT GCCCTGACCATCTCGGATGCGCAGCCTGAAGATGAGGCTGAGTATTACTGTTCG CTCTCCGACGTTGACGGTTATCTGTTCGGAGGAGGCACCCAGCTGACCGTCCTC TCCGGAATTCTAGAACAAAAGCTTATTTCTGAAGAAGACTTGGGCGGAGGAGG ATCCGGAGGCGGAGGATCAGGAGGAGGAGGATCC, (SEQ. ID NO.: 13) or an variation thereof shown as SEQ. ID. NO.: 16, was inserted into the Sac module of pBabeSacLac2 by SfiI digestion and ligation. Next the CFTR PCR product was digested with SfiI and ligated into the Lac module via PfmlI-Sfil hybrid sites. This generated the plasmid pBabe dNP138-CFTR (WT or F508de1). The nucleotide sequence for dNP138-CFTR WT is shown in SEQ. ID. NO.: 14.

Transduction:

Transducing particles for pBabeSacLac2 constructs were generated using the Phoenix Ecotropic Packaging System (Nolan laboratory, Stanford University). Phoenix-Ecotropic cells were plated at 1.3×10⁶ cells/75 cm² flask in DMEM with calf serum without antibiotics. pBabeSacLac2 DNA was transfected as described above scaled to surface area. After 24 hours, transfection complexes were removed and replaced with 8 mL of DMEM with calf serum and incubated for 48 hours at 32° C./5% CO₂. Medium was removed and filtered through Millex-HV 0.45 μm syringe filter and flash frozen in liquid nitrogen. Recipient cells were plated at 2×10⁵ cells/35 mm dish 24 hours prior to transduction. Cells were infected by adding viral supernatant and 6 μg/mL of hexadimethrine bromide and incubated for 24 hours at 32° C./5% CO₂. Cells were replated in 75 cm² flasks and screened for expression 48 hours later.

Iodide Efflux Assay:

CFTR/FAP fusion cell lines were assessed for CFTR anion transport activity by measuring the fluorescence of the halide-sensitive dye, a 6-methoxy-N-(3-sulfopropyl)quinolinium (SPQ). Cells were plated on Mattek dishes (part no p35G-1.5-14-C) 24 hours prior to iodide efflux measurement, and correctors were added to the cells after 24 hours and 24 hours prior to iodide efflux measurements. A combination of 10 μM C4+51 μM C18, or DMSO (control vehicle) was added. SPQ was loaded into cells using a hypotonic Iodide solution (above) that was diluted with H₂O 1:1 v/v, and contained 10 mM SPQ, for 20 min at 37° C. After loading, cells with SPQ they were imaged at 40× on an inverted epi-fluorescence microscope equipped for excitation at 350 nm and emission at 455 nm; images were captured every 15 seconds. Cells were perfused with buffers warmed to 37° C. For each experiment, the following solutions were serially perfused across the cells: Iodide buffer, 3 min; Nitrate buffer, 3 min; Nitrate buffer containing 10 μM Forskolin and CFTR specific potentiator, 300 nM P2 (obtained from Cystic Fibrosis Foundation Therapeutics) (6 or 8 min); and finally Iodide buffer, (2 min). Analysis was performed with Image J by analyzing mean fluorescence intensity of pre-selected cells. Data are represented as by the maximum slope of each Iodide efflux for each condition measurement.

SPQ Solutions

Iodide solution contained NaI 130 mM, Mg(NO₃)₂ 6H₂O 1 mM, Ca(NO₃) 4H₂O 1 mM, KNO₃ 4 mM, Glucose 10 mM and HEPES Hemi-Na 20 mM, pH 7.4. Nitrate solution contained NaNO₃ 130 mM, Mg(NO₃)₂ 6H₂O 1 mM, Ca(NO₃) 4H2O 1 mM, KNO₃ 4 mM, Glucose 10 mM and HEPES Hemi-Na 20 mM, pH 7.4.

Flow Cytometry

Quantification of F508de1-CFTR rescue to the cell surface was carried out by measurement of MG-11p fluorescence activity using flow cytometry. HEK293 cells stably expressing FAP-F508de1-CFTR were plated at a cell density of 1×10⁵ cells per 35 mm dish. After 12 hours, the growth media was removed and replaced with 1 mL of DMEM media containing various corrector conditions: 10 μM C4, 5 μM CFFT-002, 5 μM C18, 10 μM C4+5 μM CFFT002, 10 μM C4+5 μM C18, 5 μM CFFT-002+5 μM C18, control vehicle (DMSO) same volume as maximum used in corrector treatment. Twenty-four hours after corrector treatment, cells were removed from the dishes with Cellstripper (non-enzymatic, Cellgro) and cells were centrifuged at 500×g for 5 minutes at 4° C., then resuspended in 1 mL of PBS. 50 nM MG-11p fluorogen was added to all samples prior to measurement. Sample analysis was performed by excitation with a 640 nm laser and emission captured with 685/35 filter set. Each condition consisted of 10,000 recorded events. Data analysis was performed using FACS Diva software to obtain the mean MG fluorescence±SEM for each population. Sample size for DMSO, C4+C18 and CFFT-002+C18 n=5. C4, CFFT-002, C18 n=3. These represent experiments performed on separate days. Each sample was normalized to the mean of C18.

Immunoblotting:

Whole cell lysates from stably expressing FAP tagged CFTR cell lines were obtained by removing cells from 10 cm dishes in RIPA buffer: 150 mM NaCl, 50 mM tris HCl pH 7.5, 1% Triton X100, 1% sodium deoxycholate, 0.1% SDS and 1 tablet of PIC/10 mL RIPA buffer (Roche #11-836-153-011). FAP-CFTR WT cell lines were grown under normal conditions, FAP-F508de1-CFTR cell lines were treated with one of the following conditions for 24 hours prior to harvesting whole cell lysates: (1) Vehicle control-cell culture grade DMSO, at the volume used for correctors or corrector combinations; (2) CFFT-002 5 μM and C18 5 μM combination; (3) C4 10 μM and C18 5 μM combination. Protein concentration was determined using a BCA Protein Assay, and 50 μg of protein was separated by SDS-PAGE using a 3% stacking gel and 5% resolving gel. Proteins were transferred to PVDF-P membrane at 35V for 18 hours at 4° C. Membranes were blocked in 5% milk for 1 hour at room temperature. Membranes were incubated overnight at 4° C. and then 1 hour at room temperature in TBS-T containing aCFTR#596 (see http://www.cftrfolding.org/CFFTReagents.htm) at 1:5000. Membranes were then incubated for 1 hour at room temperature in donkey anti-mouse secondary (obtained from Jackson Immunoresearch #715-005-150) at 1:10000 and captured on film using a SUPERSIGNAL WEST DURA™ substrate kit (from ThermoScientific #34076).

Microscope Image Acquisition:

Images were acquired with NIS-Elements software using a Nikon Ti eclipse confocal microscope with Photometrics evolve EM CCD camera using 640 nm laser excitation and 700/75 nm emission filter. 40× (1.30 NA) and 60× (1.49 NA) objectives were used to image at room temperature with DMEM media. Cells were plated on Mattek dishes (part no p35G-1.5-14-C). For F508de1 expressing cell lines, cells were plated at a density of 1×10⁵ cells. 12-24 hours later, cells were treated with C4 10 μM and C18 5 μM or vehicle (DMSO) control. After 24 hours of treatment cells were imaged in the growth medium at 37° C. and 5% CO₂ using confocal microscopy with either cell impermeant (MG-11p) or permeant (MG-Ester) dye at a concentration of 50 nM. Low temperature rescue was achieved by incubating cells at 30° C. for 24 hours. Fluorescence images of F508de1-CFTR and CFTR WT using NIH ImageJ software were prepared. 10 μm scale bars are indicated on each image.

Statistics:

Statistical analysis was carried out using GRAPHPAD PRISM™ v5 software. Student's t test two tailed unpaired was performed between each corrector condition and the DMSO vehicle treated control. Statistical significance is represented as follows: p value 0.01 to 0.05—*, 0.001 to 0.01—**, 0.0001 to 0.001—***, <0.0001—****

Reagents:

Corrector compounds, C4, CFFT-002 and C18 were obtained from the CFFT panel library (see www.cftrfolding.org). Cellstripper was purchased from Mediatech (Manassas, Va.). Fluorogens, MG-11p and MG-ester were provided by the MBIC reagent chemistry group (Carnegie Mellon University).

Development of CFTR FAP Reporter Polypeptides and Their Labeling at the Cell Surface:

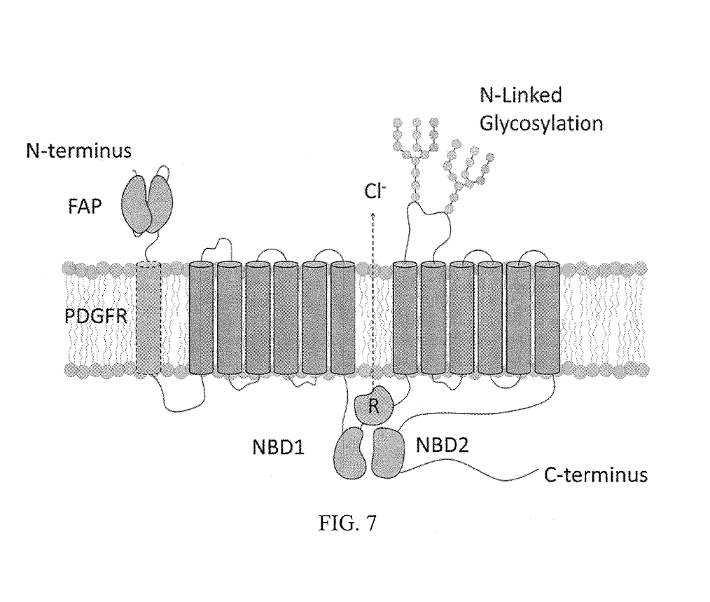

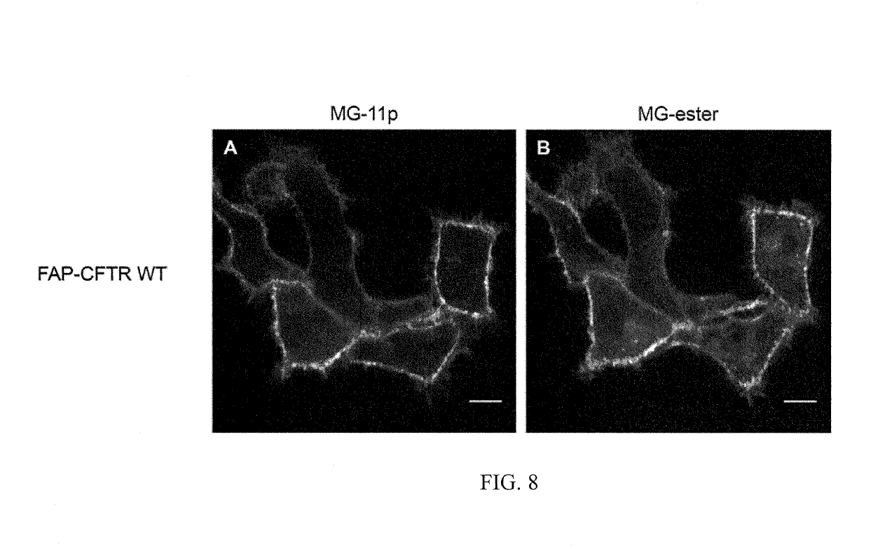

To utilize FAP based detection for CFTR, the recombinant construct described generally herein was used to tag CFTR, thus presenting the FAP portion to the extracellular environment where it is accessible to the cell impermeant fluorogen. Referring to FIG. 7, the embodiment of the recombinant construct used with CFTR was formed by modifying the N-terminus of CFTR to include the FAP via linkage to a transmembrane segment, derived from the PDGF receptor to form the construct referred to herein as FAP-CFTR.

In order to verify that the FAP constructs behaved as expected, they were expressed in HEK293 cells and the cell impermeant fluorogen, MG-11p, was used to determine whether the tagged WT CFTR had progressed to the cell surface. These cells were imaged using confocal fluorescence microscopy in the presence of either cell impermeant or cell permeant fluorogen, and representative images are provided in FIG. 8. As shown in FIG. 8A, distinct labeling of the cell membrane was observed with MG 11p, which indicates that the CFTR WT FAP fusion proteins are trafficked to, and inserted in the plasma membrane, exposing the FAP to the extracellular environment. As shown in FIG. 8B, when the same cells were exposed to MG-ester, there was a small, yet detectable, additional intracellular pool of protein, but the signal was predominantly localized to the cell surface.

Visualization of F508de1-CFTR Rescue:

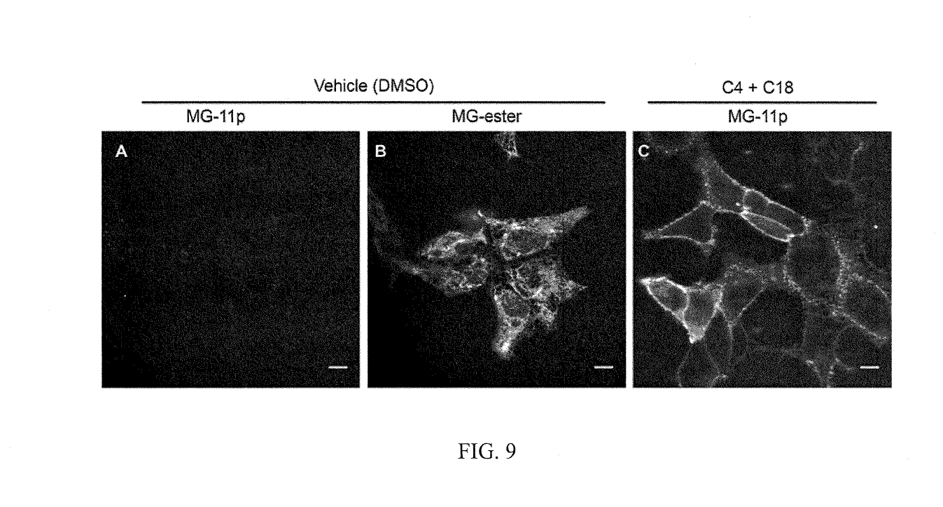

In order to study the trafficking defects of F508de1-CFTR, FAP-CFTR were used and CFTR HEK293 cells stably expressing the N-terminal constructs were grown under control conditions (vehicle, DMSO) for 24 hours and imaged with MG-11p by confocal fluorescence microscopy. As shown in FIG. 9A, there was no signal due to the added fluorogen because the F508de1-CFTR mutation prevented trafficking of the protein to the cell surface and presentation of FAP at the plasma membrane. Consequently, the FAP is not exposed to the extracellular environment and is unable to bind and activate the cell excluded MG-11p fluorogen. These images illustrate the low background level obtained from this technology, which can provide a large dynamic range for surface protein detection.

To verify fusion protein expression under normal conditions, these cells were incubated with the cell permeant fluorogen, MG-ester, which produced a significant intracellular expression pattern, shown in FIG. 9B. These observations are consistent with the trafficking defect of untagged F508de1-CFTR, as the protein is translated and inserted into the endoplasmic reticulum membrane, but translocation to and beyond the Golgi is blocked.

Rescue by Corrector Compounds:

Experiments were done to determine if the FAP fusions to F508de1-CFTR were capable of pharmacological rescue by corrector compounds. Cells were treated with a combination of correctors (C4 and C18) for 24 hours, and then imaged with MG-11p fluorogen, as shown in FIG. 9C. In contrast to the vehicle treated control (FIG. 9A), the N-terminal-CFTR expressing cell lines which were treated with a combination of correctors showed activation of the cell impermeant fluorogen. This result demonstrated that the trafficking defect of F508de1-CFTR had been rescued by corrector treatment and that the protein was present at the cell surface.

Rescue by Low Temperature Incubation:

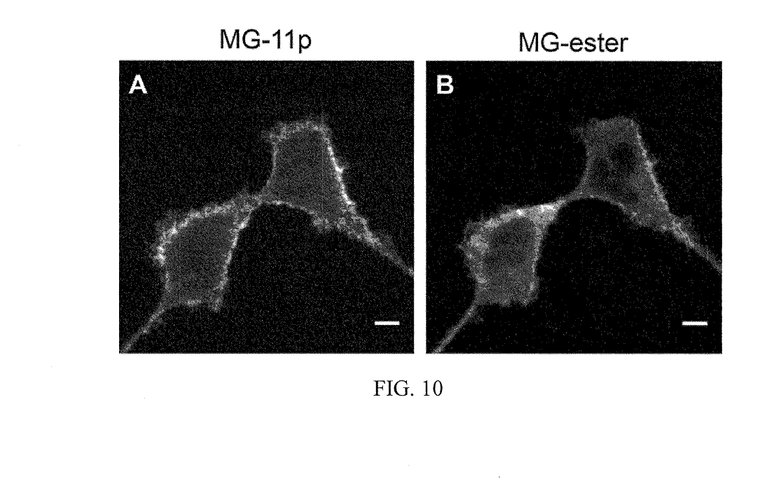

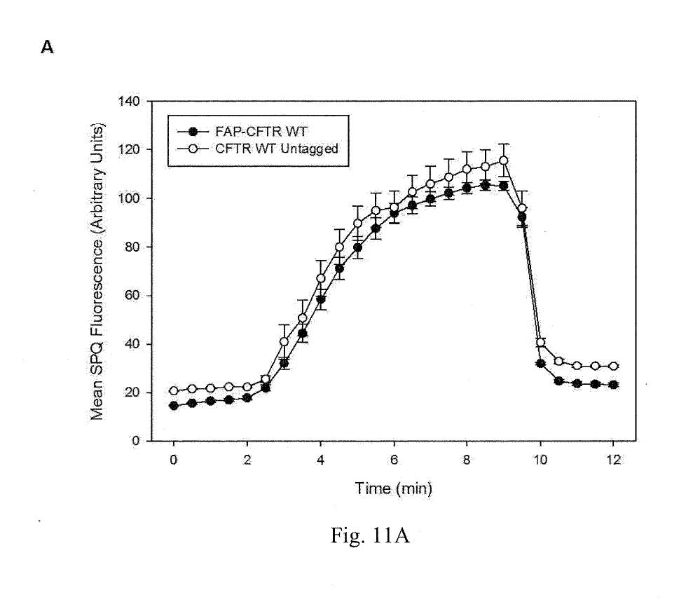

Low temperature incubation (≦30° C. for 24 h) is known to rescue the CFTR trafficking defect and allow some F508de1-CFTR to accumulate at the cell surface. HEK293 cells expressing the F508de1-CFTR FAP constructs were incubated at low temperature, exposed to cell impermeant fluorogen and cell surface labeling was detected for cells incubated at the permissive temperature (30° C.), shown in FIG. 10.

Compound Structures

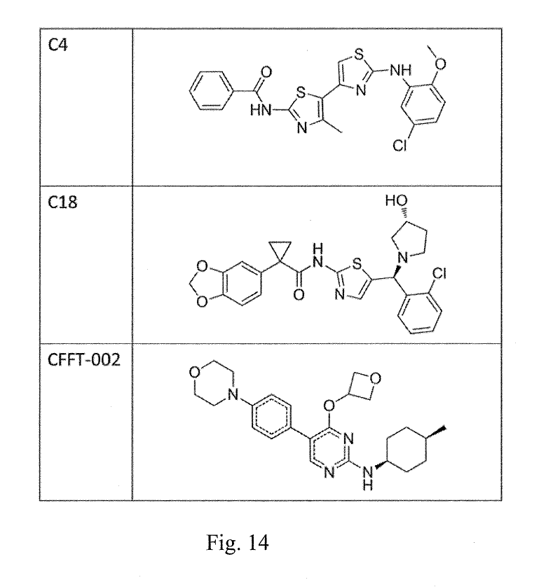

The structures of several corrector compounds are shown in FIG. 14. These corrector compounds were provided by the Cystic Fibrosis Foundation Therapeutics. Each compound was applied for 24 hours. C4 was used at 10 μM, C18 and CFFT-002 were each used at 5 μM.

Functional Rescue of F508de1-CFTR:

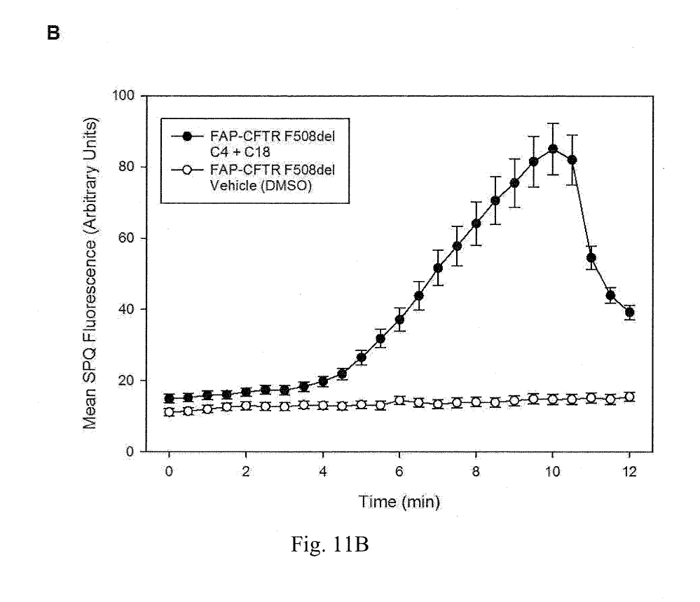

Experiments were done to determine whether the FAP interfered with CFTR-mediated cAMP-stimulated anion transport by employing a technique that measures regulated anion transport of anions across the PM using SPQ (6-methoxy-N-(3-sulfopropyl)quinolinium), a halide-sensitive fluorescent indicator dye. SPQ is quenched by iodide, so that signal is weak in iodide preloaded cells. Replacement of medium iodide with nitrate, which does not interact with SPQ, leads to dequenching of signal in cells expressing functional CFTR at the plasma membrane upon treatment with forskolin. Using this approach, it was found that the HEK293 cells expressing the N-terminus tagged CFTR WT constructs displayed a characteristic pattern of CFTR-mediated iodide efflux in response to forskolin stimulation. See FIG. 11A. The N-terminus reporter construct produced a rapid increase in SPQ fluorescence, which was consistent with that observed for untagged CFTR WT. Therefore, the cAMP stimulated anion transport activity of CFTR is maintained with the FAP modifications.

In the vehicle treated controls, the N-terminal-CFTR construct was not able to significantly increase iodide efflux in response to stimulation, as shown in FIG. 11B. In order to test whether the rescue of F508de1-CFTR FAP trafficking results in the production of functional channels, anion efflux was examined in the cell lines after treatment with C4 and C18 correctors for 24 hours. The corrector treated cells generated substantial iodide efflux in response to stimulation that was completely absent in the vehicle treated control groups, as shown in FIG. 11B. These results indicate that functional rescue of CFTR activity by corrector treatment, i.e. anion transport across the plasma membrane, occurs with the FAP tagged F508de1-CFTR constructs.

Biochemical Rescue of F508de1-CFTR:

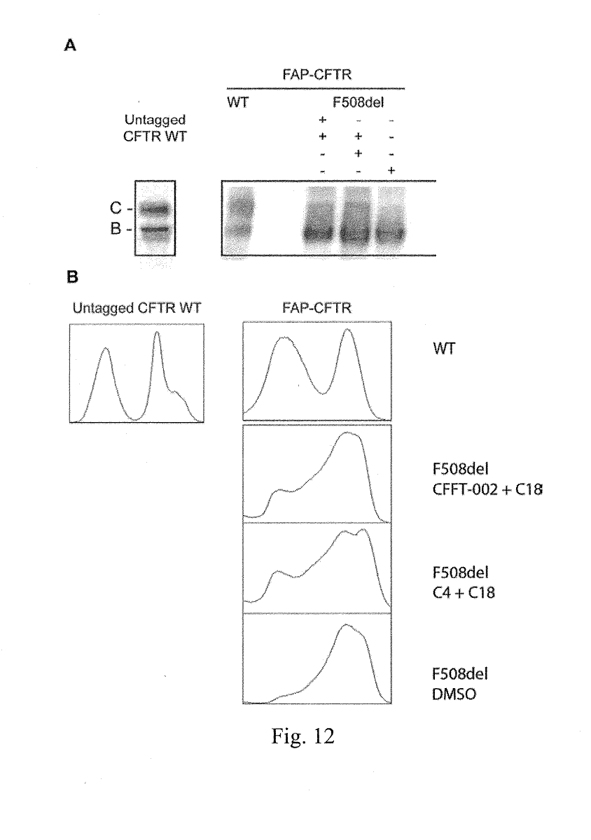

FAP-F508de1-CFTR protein expression was assessed by immunoblot analysis. Referring to FIG. 12A, the maturation of WT CFTR can be visualized as two bands on an SDS-PAGE gel: a more mobile core glycosylated form, having modifications acquired in the endoplasmic reticulum (B band) and a less mobile mature, complex glycosylated form, produced when the protein traverses the Golgi compartment (C band). Consistent with untagged WT CFTR, FAP-CFTR WT migrated as two distinct bands which are interpreted as the core and mature (B and C respectively) forms, with increases in molecular weight attributed to addition of the FAP tag (−20 kDa). Native F508de1-CFTR does not produce a mature glycosylated band because it is retained in the endoplasmic reticulum and degraded before export to the Golgi. Previous studies have shown that correctors promote the maturation of F508de1-CFTR, which can be visualized by the appearance of band C. Pedemonte, N., et al., Small-molecule correctors of defective DeltaF508-CFTR cellular processing identified by high-throughput screening, The Journal of clinical investigation, 115(9): 2564-7 (2005); Van Goor, F., et al., Rescue of DeltaF508-CFTR trafficking and gating in human cystic fibrosis airway primary cultures by small molecules, American Journal of Physiology. Lung cellular and molecular physiology, 290(6): L1117-30 (2006). To test whether the F508de1-CFTR FAP proteins also exhibit this behavior, immunoblots were performed using the CFTR specific antibody, CFFT#596. Whole cell lysates from stable HEK293 cell lines expressing the FAP-F508de1-CFTR were extracted from cells that had been treated with correctors or vehicle (DMSO). FAP-F508de1-CFTR acquired a band that migrated at the same position as the C band of FAP-CFTR WT after corrector treatment with either the CFFT-002+C18 or C4+C18 combinations. This pattern was not evident in the vehicle control lysates shown in FIG. 12A.

Quantification of Corrector Efficacy:

To gain insight on their individual properties and to characterize available correctors and combinations, flow cytometry was used to accurately quantify the effects of each corrector condition on the rescue of cell surface CFTR. Flow cytometry offers a quantitative output of the fluorescence of each cell while also providing information on the distribution of fluorescence across a large cell population. 10,000 cells per condition were analyzed and the average fluorescence intensity was taken from activation of the MG-11p signal, which reduced the effects of heterogeneity between cells due to expression differences and other factors such as cell cycle. HEK293 cell lines expressing FAP-F508de1-CFTR were incubated with single correctors or combinations of correctors at their maximal effective concentrations for 24 hours. After corrector treatment, the cells were brought into suspension using a non-enzymatic reagent to ensure that proteins at the cell surface remained intact, an issue that complicates enzymatic procedures like trypsinization. Samples were analyzed in the presence of cell impermeant fluorogen, MG-11p and the fluorescence activation across the population over three independent trials was normalized to the signal obtained for C18.

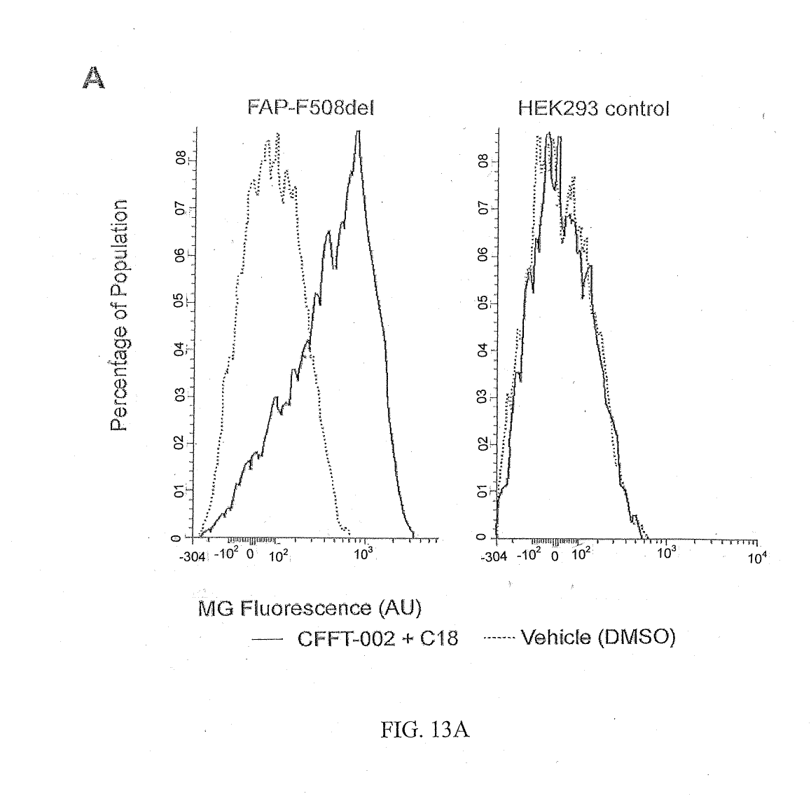

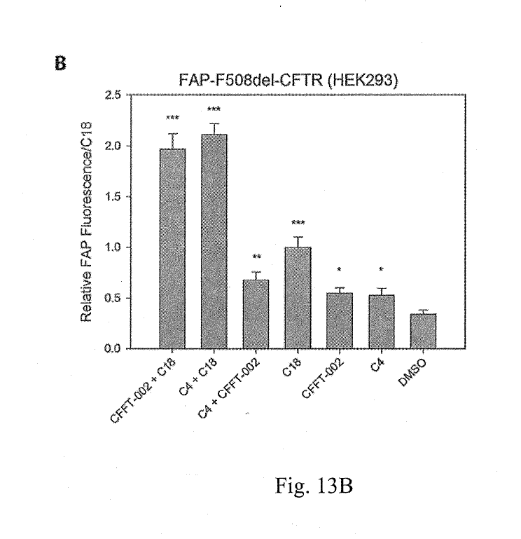

A summary of corrector efficacy data is shown in Table 1. A representative output of these experiments is shown in FIG. 13A. C18+CFFT-002 treatment produced a dramatic increase in signal in the FAP-F508de1-CFTR samples.

TABLE 1

| | FAP- |
|---|---|
| | F508del-CFTR |
| DMSO | 0.34 |
| C4 | 0.53 |
| CFFT-0002 | 0.55 |
| C18 | 1 |
| C4 + CFFT-002 | 0.68 |
| C4 + C18 | 2.11 |
| C18 + CFFT-002 | 1.97 |

### Summary of Corrector Efficacy from FAP Labeling and Functional Assays

The corrector efficacy for single compounds and combinations summarized from FIG. 13. Results are mean value normalized to C18.

FIG. 13A shows a representative flow cytometry histogram for HEK293 cell lines showing the distribution of MG-11p fluorescence signal for the vehicle control (dashed line) and CFFT-002+C18 (solid line) corrector treatment. Fluorescence activity is plotted along the horizontal axis using a logarithmic scale. FIG. 13B is a bar graph showing the mean fluorescence intensity of HEK293 cells expressing FAP-F508de1-CFTR for each condition, normalized to the effect of C18. Data are presented as the mean±SEM, n=3. Statistical significance is represented as follows: p value 0.01 to 0.05—*, 0.001 to 0.01—**, 0.0001 to 0.001—***, <0.0001—****.

The well-studied corrector, C4, produced a small improvement in plasma membrane density compared to the vehicle treated control, 0.53 vs. 0.34 for the N-terminus construct, respectively (FIG. 13B). A statistically significant difference was found for FAP-F508de1-CFTR when treated with C4. Another corrector, CFFT-002 performed similarly to C4, increasing the density of the N-terminus at the cell surface to 0.55 (FIG. 13B). The most potent single agent corrector tested was C18. Levels of trafficking correction reached 3.5 fold better than the reference corrector, C4, for the N-terminus tagged F508de1-CFTR construct after subtraction of the vehicle (DMSO) control.

Corrector Combinations

Combinations of correctors were tested to assess whether they produced more robust rescue when used together. In the FAP-F508de1-CFTR expressing cells, a combination of C4 and CFFT-002 did not statistically improve the efficacy over either compound alone (FIG. 13B). A combination of CFFT-002 and C18 however, resulted in a significant increase in surface expression resulting in an 8.6 fold increase over the rescue achieved by C4 alone. Similarly, C4 and C18 treatment produced a 9.3 fold improvement compared to C4 correction. There was no statistically significant difference found for the CFFT-002+C18 versus C4+C18 corrector conditions. Even though all of the correctors were used at their maximal effective concentrations, the FAP-F508de1-CFTR construct showed greatly improved trafficking with the corrector combinations C4+C18 or CFFT-002+C18 compared to single compound treatments (Table 1 and FIG. 13B).

Selective detection of proteins at the cell surface is a prerequisite for studying the trafficking behavior of membrane proteins, like CFTR, to and from the plasma membrane and for evaluating therapeutic approaches to diseases of protein folding. Traditionally this has been accomplished by time and labor intensive biochemical labeling methods such as biotinylation and immunofluorescence. By using a genetically encoded FAP reporter polypeptide, however, CFTR can be selectively labeled at the cell surface in live cells instantly without the need for incubation or wash steps that may modify the cellular handling of CFTR. The cell impermeant fluorogen remains dark when free in solution, however upon binding to the FAP, fluorogen fluorescence increases >15,000-fold. These features provide low background signal, excellent signal-to-noise, a large dynamic range and they eliminate the need for wash or blocking steps to remove non-specific signals. Proper trafficking and localization of FAP-tagged CFTR to the cell surface was confirmed using confocal fluorescence microscopy in stable cell lines. Importantly, F508de1-CFTR FAP protein was absent from the plasma membrane under normal conditions, but was readily detected by cell impermeant fluorogen after treatment by currently available correctors.

Immunoblot analysis was used to reveal the glycosylation state of the FAP-CFTR WT. The N-terminal FAP-CFTR WT fusion protein migrated as two distinct bands, the core and mature bands characteristic of untagged CFTR WT. Importantly, immunoblots showed that corrector treatment was capable of rescuing the maturation of FAP-F508de1-CFTR, albeit modestly (FIG. 12). Accumulation of the C band after corrector treatment is quite low with untagged F508de1-CFTR and can depend on the cell type examined. To date, C4 is one of the most well studied correctors for F508de1-CFTR rescue. The efficiency of rescue for C4 however, is quite limited across multiple cell types that have been tested. Cholon et al., Modulation of endocytic trafficking and apical stability of CFTR in primary human airway epithelial cultures, American journal of physiology. Lung cellular and molecular physiology, 2010; Varga et al., Enhanced cell-surface stability of rescued DeltaF508 cystic fibrosis transmembrane conductance regulator (CFTR) by pharmacological chaperones, The Biochemical Journal, 2008. This is in agreement with data obtained from both FAP-F508de1-CFTR constructs in HEK293 cells where C4 correction efficacy was poor (see Table 1). Unlike C4, C18 displayed a more robust rescue in both HEK293 cells expressing FAP-CFTR constructs.

Combinations of corrector treatments, particularly CFFT-002 and C18, improved FAP-F508de1-CFTR trafficking and function over single corrector treatments alone (Table 1).

As the development of combination therapies to correct CFTR trafficking may be required to obtain sufficient efficacy, the availability of a method with a broad dynamic range should optimize the detection of signal that approaches that of WT CFTR.

The development of this unique fluorescent detection platform addresses a largely unmet need in cystic fibrosis research, providing the ability to rapidly detect CFTR at the cell surface. Considerable progress has been made towards understanding the biology and physiology of CFTR; however, technological restrictions such as limited dynamic range, multiple wash and labeling steps as well as long data acquisition times may have hampered drug development. As a proof of principle, this system has been validated with known corrector compounds, but could be adapted to a multiwell format to screen for new correctors. Therefore, this fluorescence based approach could streamline the current drug development pipeline. Moreover, this system was designed in a manner that makes it generally adaptable to examine other conditions that impair protein folding and/or plasma membrane trafficking This growing list includes diseases such as type II diabetes (GLUT4, insulin receptor), long QT syndrome (hERG), familial hypercholesterolemia (LDL receptor), diabetes insipidus (aquaporin-2), diseases of retinal degeneration (BEST1) and others.

FAP Reporter Appended to Cytosolic C-Terminus

In the examples given above, the reporter and membrane-spanning linker were recombinantly linked to membrane proteins of interest at cytosolic N-termini. In the following example, a FAP reporter and membrane spanning linker would be appended to a protein of interest at a cytosolic C-terminus.

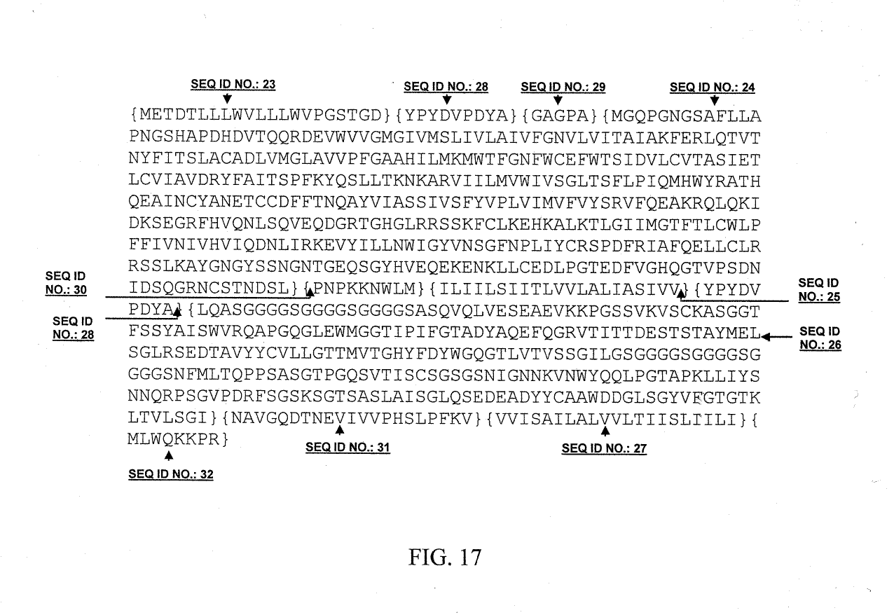

A DNA molecule that encodes the amino acid sequence for the human beta2 adrenergic receptor (SEQ. ID. NO.: 24) would be cloned by methods described herein and known in the art into the Lac stuffer site of the vector pDisplayBlue (Szent-Gyorgyi et al, 2008, supra.). A second nucleotide sequence encoding the transmembrane amino acid sequence of human PDGFRB, but with N-to-C sequence reversed relative to that of the native sequence (SEQ. ID. NO.: 25), followed by a nucleotide sequence encoding the amino acid sequence for FAP HL1.0.1-TO1 (SEQ. ID. NO.: 26), would be cloned into the BsmI site of the vector by cloning methods well known in the art. As shown in FIG. 17, the following resulting recombinant protein construct includes portions from the pDisplayBlue vector, including the Igκ leader sequence (SEQ. ID. NO.: 23), HA epitope tag sequences (SEQ. ID. NO.: 28), a transmembrane domain from human FGFRB (SEQ. ID. NO.: 27) and short sections of residues derived from the vector, shown as SEQ. ID. NOS.: 29, 30, 31, and 32.

The complete encoded recombinant protein has the sequence shown below:

(SEQ ID NO.: 22)
METDTLLLWVLLLWVPGSTGDYPYDVPDYAGAGPAMGQPGNGSAFLLAPN
GSHAPDHDVTQQRDEVWVVGMGIVMSLIVLAIVFGNVLVITAIAKFERLQ
TVTNYFITSLACADLVMGLAVVPFGAAHILMKMWTFGNFWCEFWTSIDVL
CVTASIETLCVIAVDRYFAITSPFKYQSLLTKNKARVIILMVWIVSGLTS
FLPIQMHWYRATHQEAINCYANETCCDFFTNQAYVIASSIVSFYVPLVIM
VFVYSRVFQEAKRQLQKIDKSEGRFHVQNLSQVEQDGRTGHGLRRSSKFC
LKEHKALKTLGIIMGTFTLCWLPFFIVNIVHVIQDNLIRKEVYILLNWIG
YVNSGFNPLIYCRSPDFRIAFQELLCLRRSSLKAYGNGYSSNGNTGEQSG
YHVEQEKENKLLCEDLPGTEDFVGHQGTVPSDNIDSQGRNCSTNDSLPNP
KKQWLMILIILSIITLVVLALIASIVVYPYDVPDYALQASGGGGSGGGGS
GGGGSASQVQLVESEAEVKKPGSSVKVSCKASGGTFSSYAISWVRQAPGQ
GLEWMGGTIPIFGTADYAQEFQGRVTITTDESTSTAYMELSGLRSEDTAV
YYCVLLGTTMVTGHYFDYWGQGTLVTVSSGILGSGGGGSGGGGSGGGGSN
FMLTQPPSASGTPGQSVTISCSGSGSNIGNNKVNWYQQLPGTAPKLLIYS
NNQRPSGVPDRFSGSKSGTSASLAISGLQSEDEADYYCAAWDDGLSGYVF
GTGTKLTVLSGINAVGQDTQEVIVVPHSLPFKVVVISAILALVVLTIISL
IILIMLWQKKPR.

The vector constructed as described would be transfected into HEK293 cells using standard methods, and 48 hours post-transfection the cells would be exposed to 50 nM TO1-2p fluorogen and examined by fluorescence microscopy. Cell surface fluorescence would be observed in the transfected cells, indicative of the presence of the protein in the plasma membrane with the topology shown in FIG. 1C. Further, when the transfectants are exposed to 10 μM isoproterenol, protein internalization into cytoplasmic vesicles would be observed, with the extent of internalization substantially equivalent to that observed for B₂AR with a FAP tag at its N-terminal, (See Examples above; Fisher et al., 2010), indicating that the fusion protein is physiologically responsive to agonist.

Those skilled in the art will recognize that transgenic nonhuman organisms and nonhuman mammals comprising a plurality of the aforementioned cells that contain the recombinant protein constructs encompassed by the present invention may be developed using procedures well known in the art, such as those disclosed in U.S. Pat. Nos. 4,736,866, 5,175,383, 5,175,384, 5,175,385, 5,183,949, 5,387,742, 5,589,604, and 5,639,940, wherein the transgenic animals were produced by variations of the technique wherein recombinant DNA segments are introduced into fertilized eggs of mammalian species and the embryos are implanted into surrogate mothers to obtain transgenic offspring.

This specification has been written with reference to various non-limiting and non-exhaustive embodiments. However, it will be recognized by persons having ordinary skill in the art that various substitutions, modifications, or combinations of any of the disclosed embodiments (or portions thereof) may be made within the scope of this specification. Thus, it is contemplated and understood that this specification supports additional embodiments not expressly set forth herein. Such embodiments may be obtained, for example, by combining, modifying, or reorganizing any of the disclosed steps, components, elements, features, aspects, characteristics, limitations, and the like, of the various non-limiting embodiments described in this specification.

Patents, patent applications, publications, scientific articles, books, web sites, and other documents and materials referenced or mentioned herein are indicative of the levels of skill of those skilled in the art to which the inventions pertain, as of the date each publication was written, and all are incorporated by reference as if fully rewritten herein. Inclusion of a document in this specification is not an admission that the document represents prior invention or is prior art for any purpose.

We claim:

1. A recombinant protein construct comprising:
a. a membrane protein having, in a native state, a cytosolic terminus;
b. a fusion partner comprising a reporter polypeptide; and
c. a membrane-spanning linker bound at a first end to the cytosolic terminus of the membrane protein and at a second end to the reporter polypeptide.

2. The recombinant protein construct of claim 1 wherein the membrane protein has a cytosolic N-terminus and the membrane-spanning linker is recombinantly linked to the N-terminus.

3. The recombinant protein construct of claim 1 wherein the reporter polypeptide comprises a fluorogen activating protein.

4. The recombinant protein construct of claim 3 wherein the fluorogen activating protein is selected from the group consisting of HL1-TO1, HL1.1-TO1, HL1.0.1-TO1, HL4-MG, L5-MG, H6-MG, K7, M8 and dNP138.

5. The recombinant protein construct of claim 1 wherein the reporter polypeptide comprises a fluorescent protein.

6. The recombinant protein construct of claim 1 wherein the membrane-spanning linker sequence is taken from the transmembrane domain of human platelet derived growth factor receptor beta (PDGFRB) protein.

7. The recombinant protein construct of claim 1 wherein the membrane-spanning linker has at least 85% sequence identity to the transmembrane domain of human platelet derived growth factor receptor beta (PDGFRB) protein.

8. The recombinant protein construct of claim 1 wherein the membrane-spanning linker is a polypeptide sequence having at least 85% sequence identity to a transmembrane domain selected from the group consisting of CD4TL, EGFR, VEGFR1, VGFR2, VGFR3, FGFR1-4, and TGFBR1-3.

9. The recombinant protein construct of claim 1 wherein the membrane protein is human GLUT4.

10. The recombinant protein construct of claim 1 wherein the membrane protein is human CFTR.

11. The recombinant protein construct of claim 1 wherein the membrane protein is one of human GLUT4 or human CFTR and the reporter polypeptide is a fluorogen activating protein selected from HL1.1-TO1 and HL4-MG.

12. The recombinant protein construct of claim 1 wherein the membrane protein has a cytosolic C-terminus and the membrane-spanning linker is recombinantly linked to the C-terminus.

13. The recombinant protein construct of claim 12 wherein the reporter polypeptide comprises a fluorogen activating protein.

14. The recombinant protein construct of claim 13 wherein the fluorogen activating protein is selected from the group consisting of HL1-TO1, HL1.1-TO1, HL1.0.1-TO1, HL4-MG, L5-MG, H6-MG, K7, M8 and dNP138.

15. The recombinant protein construct of claim 12 wherein the reporter polypeptide comprises a fluorescent protein.

16. The recombinant protein construct of claim 12 wherein the membrane-spanning linker has at least 85% sequence identity to the transmembrane domain of human platelet derived growth factor receptor beta (PDGFRB) protein.

17. The recombinant protein construct of claim 12 wherein the membrane-spanning linker is a domain of G-protein coupled receptors (GPCR).

18. The recombinant protein construct of claim 17 wherein the membrane protein is a human beta 2 androgenic receptor (B₂AR).

19. A nucleic acid molecule encoding the recombinant protein construct of claim 1.

20. A nucleic acid molecule encoding the recombinant protein construct of claim 9.

21. A nucleic acid molecule encoding the recombinant protein construct of claim 10.

22. A nucleic acid molecule encoding the recombinant protein construct of claim 11.

23. A nucleic acid molecule encoding the recombinant protein construct of claim 18.

24. A cell comprising the recombinant protein construct of claim 1.

24. A cell comprising the nucleic acid of claim 18.

25. A transgenic nonhuman organism comprising a plurality of the cells of claim 24.


SEQUENCE LISTING (40 sequences)

SEQ ID NO: 1 - DNA, 40 bp, Artificial Sequence
  Primer
tatataggcccagccggccccacatatacctgccgttcac

SEQ ID NO: 2 - DNA, 42 bp, Artificial Sequence
  Primer
tatataggcccctgcggccacgtcaatgccaataggatatcg

SEQ ID NO: 3 - DNA, 20 bp, Artificial Sequence
  Primer
agaggatctgaatgctgtgg

SEQ ID NO: 4 - DNA, 59 bp, Artificial Sequence
  Primer
ctcgagctaacgccacctgctggcatcgtccaggctgtggacgtggcttcttctgccaa

SEQ ID NO: 5 - DNA, 31 bp, Artificial Sequence
  Primer
ccacagcctgggttagctcactcattaggca

SEQ ID NO: 6 - DNA, 59 bp, Artificial sequence; identical to SEQ ID NO: 4
  Primer

SEQ ID NO: 7 - DNA, 31 bp, Artificial sequence; identical to SEQ ID NO: 5
  Primer

SEQ ID NO: 8 - DNA, 37 bp, Artificial Sequence
  Primer
ccacctgctggctaacgccagtttgaggggacgacga

SEQ ID NO: 9 - DNA, 13 bp, Artificial Sequence
  Primer
ggcccagccggcc

SEQ ID NO: 10 - DNA, 13 bp, Artificial Sequence
  Primer
ggccgcaggggcc

SEQ ID NO: 11 - DNA, 34 bp, Artificial Sequence
  Primer
ggcccagccggccatgcagaggtcgcctctggaa

SEQ ID NO: 12 - DNA, 33 bp, Artificial Sequence
  Primer
gcccctgcggccctaaagccttgtatcttgcac

SEQ ID NO: 13 - DNA, 894 bp, Artificial Sequence
  Malachite green (MG) binding fluorogen-activating protein (FAP)
tacccatacgacgttccagactacgctctgcaggctagtggtggtggtggttctggtggtggtggttctggtggtggtggttctgctagccaggccgtcgttacccaagaacctagtgttaccgttagcccaggtggtactgttatacttacttgtggaagtggtacgggtgccgtcacatctggtcattatgcaaattggtttcaacaaaaaccaggacaagctccaagagctttgatttttgatactgataagaagtattcttggaccccaggtagattttctggatctttgctgggagcaaaggcagctttgacaatatcagatgctcagcctgaggacgaagccgagtattactgttctcttagcgacgtggatggctacttgtttggcggtggaacacaactgacggttctgtccggtggtggcggctctggtggcggtggcagcggcggtggtggttccggaggcggcggttctcaggctgtggtgactcaggagccgtcagtgactgtgtccccaggagggacagtcattctcacttgtggctccggcactggagctgtcaccagtggtcattatgccaactggttccagcagaagcctggccaagcccccagggcacttatatttgacaccgacaagaagtattcctggacccctggccgattctcaggctccctccttggggccaaggctgccctgaccatctcggatgcgcagcctgaagatgaggctgagtattactgttcgctctccgacgttgacggttatctgttcggaggaggcacccagctgaccgtcctctccggaattctagaacaaaagcttatttctgaagaagacttgggcggaggaggatccggaggcggaggatcaggaggaggaggatcc

SEQ ID NO: 14 - DNA, 5504 bp, Artificial Sequence
  Cystic fibrosis transmembrane conductance (CFTR) linked to membrane spanning linker and fluorogen-activating protein (FAP)
atggagacagacacactcctgctatgggtactgctgctctgggttccaggttccactggtgactatccatatgatgttccagattatgctggggcccagccggcccaggccgtcgttacccaagaacctagtgttaccgttagcccaggtggtactgttatacttacttgtggaagtggtacgggtgccgtcacatctggtcattatgcaaattggtttcaacaaaaaccaggacaagctccaagagctttgatttttgatactgataagaagtattcttggaccccaggtagattttctggatctttgctgggagcaaaggcagctttgacaatatcagatgctcagcctgaggacgaagccgagtattactgttctcttagcgacgtggatggctacttgtttggcggtggaacacaactgacggttctgtccggtggtggcggctctggtggcggtggcagcggcggtggtggttccggaggcggcggttctcaggctgtggtgactcaggagccgtcagtgactgtgtccccaggagggacagtcattctcacttgtggctccggcactggagctgtcaccagtggtcattatgccaactggttccagcagaagcctggccaagcccccagggcacttatatttgacaccgacaagaagtattcctggacccctggccgattctcaggctccctccttggggccaaggctgccctgaccatctcggatgcgcagcctgaagatgaggctgagtattactgttcgctctccgacgttgacggttatctgttcggaggaggcacccagctgaccgtcctctccggccgcaggggccgggatccgcggctgcaggtcgacgaacaaaaactcatctcagaagaggatctgaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgtccacagccggccatgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgatcgagaacatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatgatgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatactgcctcgcatcagcgtgatcagcactggccccacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcttttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttagggccgcag

SEQ ID NO: 15 - DNA, 100 bp, Artificial Sequence
  Immunoglobulin kappa chain leader sequence
atggagacagacacactcctgctatgggtactgctgctctgggttccaggttccactggtgactatccatatgatgttccagattatgctggggcccagc

SEQ ID NO: 16 - DNA, 733 bp, Artificial Sequence
  fluorogen-activating protein dNP138
cggcccaggccgtcgttacccaagaacctagtgttaccgttagcccaggtggtactgttatacttacttgtggaagtggtacgggtgccgtcacatctggtcattatgcaaattggtttcaacaaaaaccaggacaagctccaagagctttgatttttgatactgataagaagtattcttggaccccaggtagattttctggatctttgctgggagcaaaggcagctttgacaatatcagatgctcagcctgaggacgaagccgagtattactgttctcttagcgacgtggatggctacttgtttggcggtggaacacaactgacggttctgtccggtggtggcggctctggtggcggtggcagcggcggtggtggttccggaggcggcggttctcaggctgtggtgactcaggagccgtcagtgactgtgtccccaggagggacagtcattctcacttgtggctccggcactggagctgtcaccagtggtcattatgccaactggttccagcagaagcctggccaagcccccagggcacttatatttgacaccgacaagaagtattcctggacccctggccgattctcaggctccctccttggggccaaggctgccctgaccatctcggatgcgcagcctgaagatgaggctgagtattactgttcgctctccgacgttgacggttatctgttcggaggaggcacccagctgaccgtcctctccggccgcag

SEQ ID NO: 17 - DNA, 215 bp, Artificial Sequence
  platelet derived growth factor receptor beta (PDGFRB)
gggccgggatccgcggctgcaggtcgacgaacaaaaactcatctcagaagaggatctgaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgtccacagc

SEQ ID NO: 18 - DNA, 4456 bp, Artificial Sequence
  Cystic fibrosis transmembrane conductance (CFTR)
cggccatgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgatcgagaacatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatgatgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatactgcctcgcatcagcgtgatcagcactggccccacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcttttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttagggccgcag

SEQ ID NO: 19 - DNA, 2726 bp, Artificial Sequence
  Insulin-regulated glucose receptor (GLUT4) linked to membrane spanning linker and fluorogen-activating protein (FAP)
atggagacagacacactcctgctatgggtactgctgctctgggttccaggttccactggtgactatccatatgatgttccagattatgctggggcccagccggcctacccatacgacgttccagactacgctctgcaggctagtggtggtggtggttctggtggtggtggttctggtggtggtggttctgctagccaggtgcagctggtggaatctgaggctgaggtgaagaagcctgggtcctcggtgaaggtctcctgcaaggcttctggaggcaccttcagcagctatgctatcagctgggtgcgacaggcccctggacaagggctcgagtggatgggagggaccatccctatctttggtacagcaaactacgcacagaagttccagggcagagtcacgattaccgcggacgagtccacgagcacagcctacatggagctgagcagcctgagatctgaggacacggccgtgtattactgtgtcttgttgggtacaactatggttacgggatactactttgactactggggccagggaaccctggtcaccgtctcctcaggaattctaggatccggtggcggtggcagcggcggtggtggttccggaggcggcggttctaattttacgctgactcagcccccctcagcgtctgggacccccgggcagagcgtcaccatctcttgttctggaagcggctcgaacatcggaaacaataaagtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggttatgtcttcggaactgggaccaagctcaccgtcctatccggaattggccgcaggggccgggatccgcggctgcaggtcgacgaacaaaaactcatctcagaagaggatctgaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgtccacagccggccatgccgtcgggcttccaacagataggctccgaagatggggaaccccctcagcagcgagtgactgggaccctggtccttgctgtgttctctgcggtgcttggctccctgcagtttgggtacaacattggggtcatcaatgcccctcagaaggtgattgaacagagctacaatgagacgtggctggggaggcaggggcctgagggacccagctccatccctccaggcaccctcaccaccctctgggccctctccgtggccatcttttccgtgggcggcatgatttcctccttcctcattggtatcatctctcagtggcttggaaggaaaagggccatgctggtcaacaatgtcctggcggtgctggggggcagcctcatgggcctggccaacgctgctgcctcctatgaaatgctcatccttggacgattcctcattggcgcctactcagggctgacatcagggctggtgcccatgtacgtgggggagattgctcccactcacctgcggggcgccctggggacgctcaaccaactggccattgttatcggcattctgatcgcccaggtgctgggcttggagtccctcctgggcactgccagcctgtggccactgctcctgggcctcacagtgctacctgccctcctgcagctggtcctgctgcccttctgtcccgagagcccccgctacctctacatcatccagaatctcgaggggcctgccagaaagagtctgaagcgcctgacaggctgggccgatgtttctggagtgctggctgagctgaaggatgagaagcggaagctggagcgtgagcggccactgtccctgctccagctcctgggcagccgtacccaccggcagcccctgatcattgcggtcgtgctgcagctgagccagcagctctctggcatcaatgctgttttctattattcgaccagcatcttcgagacagcaggggtaggccagcctgcctatgccaccataggagctggtgtggtcaacacagtcttcaccttggtctcggtgttgttggtggagcgggcggggcgccggacgctccatctcctgggcctggcgggcatgtgtggctgtgccatcctgatgactgtggctctgctcctgctggagcgagttccagccatgagctacgtctccattgtggccatctttggcttcgtggcattttttgagattggccctggccccattccttggttcatcgtggccgagctcttcagccagggaccccgcccggcagccatggctgtggctggtttctccaactggacgagcaacttcatcattggcatgggtttccagtatgttgcggaggctatggggccctacgtcttccttctatttgcggtcctcctgctgggcttcttcatcttcaccttcttaagagtacctgaaactcgaggccggacgtttgaccagatctcagctgccttccaccggacaccctctcttttagagcaggaggtgaaacccagcacagaacttgagtatttagggccagatgagaacgactgaggccgcag

SEQ ID NO: 20 - DNA, 868 bp, Artificial Sequence
  fluorogen-activating protein T01.1
cggcctacccatacgacgttccagactacgctctgcaggctagtggtggtggtggttctggtggtggtggttctggtggtggtggttctgctagccaggtgcagctggtggaatctgaggctgaggtgaagaagcctgggtcctcggtgaaggtctcctgcaaggcttctggaggcaccttcagcagctatgctatcagctgggtgcgacaggcccctggacaagggctcgagtggatgggagggaccatccctatctttggtacagcaaactacgcacagaagttccagggcagagtcacgattaccgcggacgagtccacgagcacagcctacatggagctgagcagcctgagatctgaggacacggccgtgtattactgtgtcttgttgggtacaactatggttacgggatactactttgactactggggccagggaaccctggtcaccgtctcctcaggaattctaggatccggtggcggtggcagcggcggtggtggttccggaggcggcggttctaattttacgctgactcagcccccctcagcgtctgggacccccgggcagagcgtcaccatctcttgttctggaagcggctcgaacatcggaaacaataaagtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggttatgtcttcggaactgggaccaagctcaccgtcctatccggaattggccgcag

SEQ ID NO: 21 - DNA, 1543 bp, Artificial Sequence
  Insulin-regulated glucose receptor (GLUT4)
cggccatgccgtcgggcttccaacagataggctccgaagatggggaaccccctcagcagcgagtgactgggaccctggtccttgctgtgttctctgcggtgcttggctccctgcagtttgggtacaacattggggtcatcaatgcccctcagaaggtgattgaacagagctacaatgagacgtggctggggaggcaggggcctgagggacccagctccatccctccaggcaccctcaccaccctctgggccctctccgtggccatcttttccgtgggcggcatgatttcctccttcctcattggtatcatctctcagtggcttggaaggaaaagggccatgctggtcaacaatgtcctggcggtgctggggggcagcctcatgggcctggccaacgctgctgcctcctatgaaatgctcatccttggacgattcctcattggcgcctactcagggctgacatcagggctggtgcccatgtacgtgggggagattgctcccactcacctgcggggcgccctggggacgctcaaccaactggccattgttatcggcattctgatcgcccaggtgctgggcttggagtccctcctgggcactgccagcctgtggccactgctcctgggcctcacagtgctacctgccctcctgcagctggtcctgctgcccttctgtcccgagagcccccgctacctctacatcatccagaatctcgaggggcctgccagaaagagtctgaagcgcctgacaggctgggccgatgtttctggagtgctggctgagctgaaggatgagaagcggaagctggagcgtgagcggccactgtccctgctccagctcctgggcagccgtacccaccggcagcccctgatcattgcggtcgtgctgcagctgagccagcagctctctggcatcaatgctgttttctattattcgaccagcatcttcgagacagcaggggtaggccagcctgcctatgccaccataggagctggtgtggtcaacacagtcttcaccttggtctcggtgttgttggtggagcgggcggggcgccggacgctccatctcctgggcctggcgggcatgtgtggctgtgccatcctgatgactgtggctctgctcctgctggagcgagttccagccatgagctacgtctccattgtggccatctttggcttcgtggcattttttgagattggccctggccccattccttggttcatcgtggccgagctcttcagccagggaccccgcccggcagccatggctgtggctggtttctccaactggacgagcaacttcatcattggcatgggtttccagtatgttgcggaggctatggggccctacgtcttccttctatttgcggtcctcctgctgggcttcttcatcttcaccttcttaagagtacctgaaactcgaggccggacgtttgaccagatctcagctgccttccaccggacaccctctcttttagagcaggaggtgaaacccagcacagaacttgagtatttagggccagatgagaacgactgaggccgcag

SEQ ID NO: 22 - protein, 812 aa, Artificial Sequence
  Human beta2 adrenergic receptor linked to membrane spanning linker and fluorogen-activating protein (FAP)
METDTLLLWVLLLWVPGSTGDYPYDVPDYAGAGPAMGQPGNGSAFLLAPNGSHAPDHDVTQQRDEVWVVGMGIVMSLIVLAIVFGNVLVITAIAKFERLQTVTNYFITSLACADLVMGLAVVPFGAAHILMKMWTFGNFWCEFWTSIDVLCVTASIETLCVIAVDRYFAITSPFKYQSLLTKNKARVIILMVWIVSGLTSFLPIQMHWYRATHQEAINCYANETCCDFFTNQAYVIASSIVSFYVPLVIMVFVYSRVFQEAKRQLQKIDKSEGRFHVQNLSQVEQDGRTGHGLRRSSKFCLKEHKALKTLGIIMGTFTLCWLPFFIVNIVHVIQDNLIRKEVYILLNWIGYVNSGFNPLIYCRSPDFRIAFQELLCLRRSSLKAYGNGYSSNGNTGEQSGYHVEQEKENKLLCEDLPGTEDFVGHQGTVPSDNIDSQGRNCSTNDSLPNPKKNWLMILIILSIITLVVLALIASIVVYPYDVPDYALQASGGGGSGGGGSGGGGSASQVQLVESEAEVKKPGSSVKVSCKASGGTFSSYAISWVRQAPGQGLEWMGGTIPIFGTADYAQEFQGRVTITTDESTSTAYMELSGLRSEDTAVYYCVLLGTTMVTGHYFDYWGQGTLVTVSSGILGSGGGGSGGGGSGGGGSNFMLTQPPSASGTPGQSVTISCSGSGSNIGNNKVNWYQQLPGTAPKLLIYSNNQRPSGVPDRFSGSKSGTSASLAISGLQSEDEADYYCAAWDDGLSGYVFGTGTKLTVLSGINAVGQDTNEVIVVPHSLPFKVVVISAILALVVLTIISLIILIMLWQKKPR

SEQ ID NO: 23 - protein, 21 aa, Artificial Sequence
  Immunoglobulin kappa chain leader sequence
METDTLLLWVLLLWVPGSTGD

SEQ ID NO: 24 - protein, 412 aa, Artificial Sequence
  Human beta2 adrenergic receptor
MGQPGNGSAFLLAPNGSHAPDHDVTQQRDEVWVVGMGIVMSLIVLAIVFGNVLVITAIAKFERLQTVTNYFITSLACADLVMGLAVVPFGAAHILMKMWTFGNFWCEFWTSIDVLCVTASIETLCVIAVDRYFAITSPFKYQSLLTKNKARVIILMVWIVSGLTSFLPIQMHWYRATHQEAINCYANETCCDFFTNQAYVIASSIVSFYVPLVIMVFVYSRVFQEAKRQLQKIDKSEGRFHVQNLSQVEQDGRTGHGLRRSSKFCLKEHKALKTLGIIMGTFTLCWLPFFIVNIVHVIQDNLIRKEVYILLNWIGYVNSGFNPLIYCRSPDFRIAFQELLCLRRSSLKAYGNGYSSNGNTGEQSGYHVEQEKENKLLCEDLPGTEDFVGHQGTVPSDNIDSQGRNCSTNDSL

SEQ ID NO: 25 - protein, 21 aa, Artificial Sequence
  Human fibroblast growth factor receptor beta transmembrane domain 1
ILIILSIITLVVLALIASIVV

SEQ ID NO: 26 - protein, 276 aa, Artificial Sequence
  Fluorogen-activating protein HL1.0.1-T01
LQASGGGGSGGGGSGGGGSASQVQLVESEAEVKKPGSSVKVSCKASGGTFSSYAISWVRQAPGQGLEWMGGTIPIFGTADYAQEFQGRVTITTDESTSTAYMELSGLRSEDTAVYYCVLLGTTMVTGHYFDYWGQGTLVTVSSGILGSGGGGSGGGGSGGGGSNFMLTQPPSASGTPGQSVTISCSGSGSNIGNNKVNWYQQLPGTAPKLLIYSNNQRPSGVPDRFSGSKSGTSASLAISGLQSEDEADYYCAAWDDGLSGYVFGTGTKLTVLSGI

SEQ ID NO: 27 - protein, 21 aa, Artificial Sequence
  Human fibroblast growth factor receptor beta transmembrane domain 2
VVISAILALVVLTIISLIILI

SEQ ID NO: 28 - protein, 9 aa, Artificial Sequence
  Hyaluronic acid (HA) epitope
YPYDVPDYA

SEQ ID NO: 29 - protein, 5 aa, Artificial Sequence
  plasmid sequence
GAGPA

SEQ ID NO: 30 - protein, 9 aa, Artificial Sequence
  Plasmid sequence
PNPKKNWLM

SEQ ID NO: 31 - protein, 21 aa, Artificial Sequence
  Plasmid sequence
NAVGQDTNEVIVVPHSLPFKV

SEQ ID NO: 32 - protein, 8 aa, Artificial Sequence
  Plasmid sequence
MLWQKKPR

SEQ ID NO: 33 - protein, 252 aa, Artificial Sequence
  fluorogen-activating protein (FAP) HL1-T01
QVQLVESEAEVKKPGSSVKVSCKASGGTFSSYAISWVRQAPGQGLEWMGGIIPIFGTANYAQKFQGRVTITADESTSTAYMELSSLRSEDTAVYYCVLLDTTMVTGYYFDYWGQGTLVTVSSGILGSGGGGSGGGGSGGGGSNFMLTQPPSASGTPGQSVTISCSGSGSNIGNNKVNWYQQLPGTAPKLLIYSNNQRPSGVPDRFSGSKSGTSASLAISGLQSEDEADYYCAAWDDSLNGYVFGTGTKLTVL

SEQ ID NO: 34 - protein, 252 aa, Artificial Sequence
  Fluorogen-activating protein HL1.1-T01
QVQLVESEAEVKKPGSSVKVSCKASGGTFSSYAISWVRQAPGQGLEWMGGTIPIFGTANYAQKFQGRVTITADESTSTAYMELSSLRSEDTAVYYCVLLGTTMVTGYYFDYWGQGTLVTVSSGILGSGGGGSGGGGSGGGGSNFTLTQPPSASGTPGQSVTISCSGSGSNIGNNKVNWYQQLPGTAPKLLIYSNNQRPSGVPDRFSGSKSGTSASLAISGLQSEDEADYYCAAWDDSLNGYVFGTGTKLTVL

SEQ ID NO: 35 - protein, 264 aa, Artificial Sequence
  Fluorogen-activating protein HL4-MG
QVQLVESEGGLVQPGGSLRLSCAASGFTFSSYEMNWVRQAPGKGLEWVSRIDGDGSSTNYADSVKGRFTISRDNAKSTLYLQMNSLRAEDTAVYYCTRARYFGSVSPYGMDVWGQGTTVTVSSGILGSGGGGSGGGGSGGGGSDIRVTQSPSSVSASVGDRVTISCRASQGIATWLGWYQQKPGKPPQLLIYSASTLQTGVPSRFSGSGSGTDFTLTISSLQPEDVATYYCQEGSTFPLTFGGGTKVDIKSGILEQKLISEEDL

SEQ ID NO: 36 - protein, 123 aa, Artificial Sequence
  Fluorogen-activating protein (FAP) L5-MG
EAEAYQAVVTQEPSVTVSPGGTVILTCGSSTGAVTSGHYANWFQQKPGQAPRALIFETDKKYPWTPGRFSGSLLGVKAALTISDAQPEDEAEYYCLLSDVDGYLFGGGTQLTVLSTGHHHHHH

SEQ ID NO: 37 - protein, 155 aa, Artificial Sequence
  Fluorogen-activating protein (FAP) H6-MG
QVQLQESGPGLVKPSETLSLTCTVSGASISSSITYYWGWIRQPPGKGPEWIGSMYYSGRTYYNPALKSRVTISPDKSKNQFFLKLTSVTAADTAVYYCAREGPTHYYDNSGPIPSDEYFQHWGQGTLVTVSSGILGSGGGGSGGGGSGGGGLQEF

SEQ ID NO: 38 - protein, 242 aa, Artificial Sequence
  Fluorogen-activating protein (FAP) K7
QVQLVQSGAEVKKPGASVKVSCKVSGYTLSESPMHWVRQAPGKGLEWMGHFDPEDGEKIYAQKFQGRVTLTDDTSTDTVYLELSSLTSEDTAVYYCATETGWGPGTLVTVSSGILGSGGGGSGGGGSGGGGSNFMLTQPRSVSESPGKTVTISCTRSSGSIASNYVQWYQQRPGSAPTTIIYEDHQRPSGVPDRFSGSIDSSSNSASLTIAGLRTEDEADYFCQSYDANDVVFGGGTKLTVL

SEQ ID NO: 39 - protein, 262 aa, Artificial Sequence
  Fluorogen-activating protein (FAP) M8
QVQLVESEGGLVQPGGSLRLSCAASGFTFSSYWMSWVRQAPGKGLEGVATIKQDGSEKYYVDSVKGRITISRDNAKNSLNRQINSLRAEDTAVYYCARDRLVRETGGDYRGLDLWGQGTTVTVSSASTKGPSGILGSGGGGSGGGGSGGGGSQPVLTQSPSVSGTPGQKVTIFCSGSSSNVEDNSVYWYQQFPGTTPKVLIYNDDRRSSGVPDRFSGSKSGTSASLAISGLRSEDEADYYCLSWDDSLNGWVFGGGTKVTVL

SEQ ID NO: 40 - protein, 241 aa, Artificial Sequence
  Fluorogen-activating protein (FAP) dNP138
ASQAVVTQEPSVTVSPGGTVILTCGSGTGAVTSGHYANWFQQKPGQAPRALIFDTDKKYSWTPGRFSGSLLGAKAALTISDAQPEDEAEYYCSLSDVDGYLFGGGTQLTVLSGGGGSGGGGSGGGGSGGGGSQAVVTQEPSVTVSPGGTVILTCGSGTGAVTSGHYANWFQQKPGQAPRALIFDTDKKYSWTPGRFSGSLLGAKAALTISDAQPEDEAEYYCSLSDVDGYLFGGGTQLTVL